(19) United States
(12) Patent Application Publication
(10) Pub. No.: US 2011/0092966 A1
(43) Pub. Date: Apr. 21, 2011

(54) ULTRASHORT PULSE LASER APPLICATIONS

(76) Inventors: Zhixiong GUO, Piscataway, NJ (US); Michael Schuler, Edison, NJ (US); Huan Huang, Piscataway, NJ (US); Xiaoliang Wang, Piscataway, NJ (US)

(21) Appl. No.: 12/906,743

(22) Filed: Oct. 18, 2010

Related U.S. Application Data
(63) Continuation of application No. PCT/US2009/040996, filed on Apr. 17, 2009.
(60) Provisional application No. 61/045,949, filed on Apr. 17, 2008.

(51) Int. Cl. A61B 18/20

(52) U.S. Cl. 606/13

(57) ABSTRACT

The invention relates to methods of processing biological tissue using an ultrashort pulse (USP) laser. In one embodiment, the invention relates to a method of separating transverse layers or portions of a biological tissue using USP laser. In an alternative embodiment, the invention relates to a method of cutting biological tissue using USP laser. In another embodiment, the invention relates to a method of removing unwanted material from the surface of a biological tissue comprising application of the USP laser to the tissue surface.

### CROSS-REFERENCE TO RELATED APPLICATIONS

This application claims the benefit of priority of U.S. Provisional Application Ser. No. 61/045,949, filed Apr. 17, 2008, which is hereby incorporated by reference in its entirety.

### BACKGROUND OF THE INVENTION

Allograft, xenograft, or autograft tissues require processing before they can be transplanted into a patient or subject. These processing methods include preparing the tissues by cutting and shaping the tissues into a form appropriate for implantation, or removing unwanted materials from its surface.

For example, allograft, xenograft, and autograft tissues often have to be modified into a particular form before implantation. This includes separating or removing layers of the tissue, or cutting the layer into a specific size or shape. For instance, the tissue may have to be separated into layers, as the tissue in its entirety may not be necessary or appropriate for implantation. In treatment of burn wounds, it may be necessary only to implant the epidermal layer of a skin allograft.

However, the field lacks an effective method for separating or removing layers of biological tissue, or for cutting and shaping the tissue. Techniques using a mechanical cutter or surgical knife to separate a tissue into layers or cut the tissue into portions are often imprecise and can result in damage to the underlying layers or surrounding tissue, respectively. These instruments also tend to be wasteful, as tissue is lost due to the width of the blade or cutters. Traditional continuous wave lasers can be used to remove or separate layers of tissue or cut tissue into portions, but these lasers can generate substantial heat during application, which can be transferred to the surrounding tissue and may result in melting or charring of the tissue. Thus, it is useful in the art for a means to precisely and safely modify allograft, xenograft, and autograft tissues as preparation for implantation.

Furthermore, removal of unwanted materials, especially contaminants, from the surface of allograft, xenograft, and autograft tissues is important for preparing the tissue for implantation. However, there are few methods that can effectively remove unwanted material without harming or damaging the tissue. Common techniques such as applying solutions comprising peracetic acid, povidone-iodine, or mixtures of antibiotics can vary in efficacy. Moreover, gamma irradiation can alter the structural and biomechanical properties of the tissue; for example, irradiation of patellar tendon grafts may reduce the biomechanical strength of the tendon, while irradiation of skin grafts may induce cross-linking of the skin matrix and cause the graft to stiffen. Therefore, it would be useful to develop an effective method of removing unwanted materials and contaminants from the surface of allograft, xenograft, and autograft tissues without damaging or altering the properties of the tissue.

### SUMMARY OF THE INVENTION

The instant invention relates to methods of processing biological tissue using an ultrashort pulse (USP) laser. In one embodiment, the invention relates to a method of separating transverse layers or portions of a biological tissue using USP laser. In an alternative embodiment, the invention relates to a method of cutting biological tissue using USP laser. In another embodiment, the invention relates to a method of removing unwanted material from the surface of a biological tissue comprising application of the USP laser to the tissue surface.

In certain embodiments, the invention relates to a method of separating a transverse layer from a biological tissue without damaging the surface of the transverse layer, comprising applying a USP laser to the tissue. In certain embodiments, the USP laser is applied in a direction normal to the surface of the transverse laser. In other embodiments, the USP laser is applied in a direction parallel to the surface of the transverse layer. In certain embodiments, the pulses of the laser have a duration of about 100 fs to about 50 ps, a repetition rate of about 1 Hz to about 500 kHz, a pulse energy of about 1 to about 100 μJ, and a wavelength of between about 776 nm and 1552 nm.

In yet other embodiments, the instant invention relates to a method of separating a transverse layer from a biological tissue without damaging the surface of the transverse layer, comprising applying a USP laser to the tissue, which further comprises focusing the USP laser to the biological tissue at a first site, wherein the focused laser induces optical breakdown and ablates at a depth below the transverse layer at the first site, and repeating the application of the focused laser to the biological tissue at a plurality of sites across the biological tissue, wherein the focused laser induces optical breakdown and ablates below the entire transverse layer.

In further embodiments, the methods of the invention further comprise applying a diagnostic laser to the biological tissue to determine the depth below the transverse layer. In some embodiments, the depth to which the laser beam of ultrashort pulses is applied and the depth below the transverse layer determined by the diagnostic laser is essentially the same.

In certain embodiments, the biological tissue employed in the methods of the present invention is selected from the group consisting of allograft, xenograft, autograft, and biologic matrix. Examples of suitable allograft, xenograft, or autograft include tissue, musculoskeletal tissue, cardiovascular tissue, connective tissue, and neural tissue. In particular embodiments, the allograft, xenograft, or autograft is dermal tissue. In further embodiments, the separated transverse layer is the epidermis. In other embodiments, the separated transverse layer is the dermis.

In certain embodiments, the biologic matrix employed in the methods of the present invention is an acellular dermal matrix.

In certain embodiments, the biological tissue employed in the methods of the present invention is selected from bone, muscle, fascia, bladder, stomach, heart, small intestine, large intestine, and parenchymal organs.

In other embodiments, the invention relates to a method of separating a transverse layer from a biological tissue without damaging the surface of the transverse layer, comprising: (i) providing a biological tissue having a surface and a transverse layer essentially parallel to the surface; (ii) generating a laser beam of ultrashort pulses, wherein the pulses have a duration of about 100 fs to about 50 ps, a repetition rate of about 1 Hz to about 500 kHz, a pulse energy of about 1 to about 100 μJ, and a wavelength of between about 776 nm and 1552 nm; (iii) applying and focusing the beam to the biological tissue at a first site, wherein the beam is in a direction normal to the transverse layer, and wherein the focused beam induces optical breakdown and ablates at a depth below the transverse layer at the first site; and (iv) repeating the application of the focused beam to the biological tissue at a plurality of sites across the biological tissue, wherein the focused beam induces optical breakdown and ablates below the entire transverse layer, thereby separating the transverse layer from the biological tissue.

In yet other embodiments, the invention relates to a method of precision separating a transverse layer from a biological tissue without damaging the surface of the transverse layer or the tissue surrounding the separated layer, comprising applying a USP laser to the tissue. In certain further embodiments, the method further comprises: (i) generating a laser beam of USP, wherein the pulses have a duration of about 100 fs to about 50 ps, a repetition rate of about 1 Hz to about 500 kHz, a pulse energy of about 1 to about 100 μJ, and a wavelength of between about 776 nm and 1552 nm; (ii) focusing the beam to the biological tissue at a first site, wherein the focused beam induces optical breakdown and ablates at a depth below the transverse layer at the first site; and (iii) repeating the application of the focused beam to the biological tissue at a plurality of sites across the biological tissue, wherein the focused beam induces optical breakdown and ablates below the entire transverse layer. In some embodiments, the USP laser is applied in a direction normal to the surface of the transverse laser. In other embodiments, the USP laser is applied in a direction parallel to the surface of the transverse layer. In certain further embodiments, the method further comprises applying a diagnostic laser to the biological tissue to determine the depth below the transverse layer. In yet other embodiments, the depth to which the laser beam of ultrashort pulses is applied and the depth below the transverse layer determined by the diagnostic laser is essentially the same.

In yet other embodiments, the invention relates to a method of cutting a biological tissue, comprising applying a USP laser to the tissue. In certain embodiments, the pulses have a duration of about 100 fs to about 50 ps, a repetition rate of about 1 Hz to about 500 kHz, a pulse energy of about 1 to about 100 μJ, and a wavelength of between about 776 nm and 1552 nm. In further embodiments, the method further comprises applying the USP laser to the tissue until the tissue is separated in two or more portions. In certain embodiments, the method further comprises (i) focusing the beam to the biological tissue at different depths, wherein the focused beam induces optical breakdown and ablates the biological tissue at the focused site; (ii) repeating the application of the focused beam to the biological tissue in a plurality of sites through the depth of the biological tissue, wherein the focused beam ablates the biological tissue at the plurality of sites.

In other embodiments, the subject invention relates to a method of cutting a biological tissue, comprising: (i) providing a biological tissue; (ii) generating a laser beam of ultrashort pulses, wherein the pulses have a duration of about 100 fs to about 50 ps, a repetition rate of about 1 Hz to about 500 kHz, a pulse energy of about 1 to about 100 μJ, and a wavelength of between about 776 nm and 1552 nm; (iii) applying and focusing the beam to the biological tissue at different depths, wherein the beam is in a direction normal to the surface, and wherein the focused beam induces optical breakdown and ablates the biological tissue at the focused site; (iv) repeating the application of the focused beam to the biological tissue in a plurality of sites through the depth of the biological tissue, wherein the focused beam ablates the biological tissue at the plurality of sites, thereby cutting the tissue.

In other embodiments, the invention relates to a method of precision cutting a biological tissue, comprising applying a USP laser to the tissue which does not induce damage to the tissue surrounding the cut. In yet other embodiments, the method further comprises applying the USP laser to the tissue until the tissue is separated in two or more portions. In further embodiments, the method comprises: (i) generating a laser beam of ultrashort pulses, wherein the pulses have a duration of about 100 fs to about 50 ps, a repetition rate of about 1 Hz to about 500 kHz, a pulse energy of about 1 to about 100 μJ, and a wavelength of between about 776 nm and 1552 nm; (ii) applying and focusing the beam to the biological tissue at different depths, wherein the beam is in a direction normal to the surface, and wherein the focused beam induces optical breakdown and ablates the biological tissue at the focused site; and (iii) repeating the application of the focused beam to the biological tissue in a plurality of sites through the depth of the biological tissue, wherein the focused beam ablates the biological tissue at the plurality of sites.

In other embodiments, the invention relates to a method of ablating unwanted material from an area on a surface of a biological tissue, comprising applying a USP laser to the surface of the tissue. In certain embodiments, the USP laser is applied in a direction normal to the surface of the transverse laser. In other embodiments, the USP laser is applied in a direction parallel to the surface of the transverse layer. In certain embodiments, the pulses of the laser have a duration of about 100 fs to about 50 ps, a repetition rate of about 1 Hz to about 500 kHz, a pulse energy of about 1 to about 100 μJ, and a wavelength of between about 776 nm and 1552 nm. In further embodiments, the method further comprises focusing the beam to the surface of the biological tissue at a first site with a focus spot size in the range of 2-10 μm, wherein the beam is to a depth below the unwanted material, and wherein the focused beam induces optical breakdown and removes the unwanted material at the first site via laser-induced plasma ablation, and repeating the application of the focused beam to the surface of the biological tissue at a plurality of sites across the surface of the biological tissue, wherein: (a) the focused beam ablates the unwanted material at the plurality of sites, (b) the plurality of sites are adjacent to each other, and (c) the plurality of sites form an area. In certain embodiments, the method further comprises applying a diagnostic laser beam to the surface of the biological tissue to determine the depth of the unwanted material. In some embodiments, the depth to which the laser beam of ultrashort pulses is applied and the depth of the unwanted material determined by the diagnostic laser is essentially the same.

Examples of unwanted material that may be ablated from an area on a surface of a biological tissue according to the methods described herein include gram positive bacteria, gram negative bacteria, spore-forming bacteria, yeasts, and fungi. Examples of gram positive bacteria include Clostridium spp, Aerococcus, Micrococcus, Staphylococcus aureus, Staphylococcus sciuri, Staphylococcus epidermidis, and Bacillus cereus. Examples of gram negative bacteria include Acinetobacter or E. coli.

In some embodiments, the unwanted material that may be ablated according to the methods of the present invention include a layer of cells. In certain embodiments, the layer of cells are dermal cells.

In other embodiments, the unwanted material comprises residual skin hairs. In certain embodiments, the unwanted material further comprises hair follicles. In other embodiments, the unwanted material further comprises the hair shaft.

In certain embodiments, the invention relates to a method of ablating unwanted material from an area on a surface of a biological tissue, comprising: (i) providing a biological tissue; (ii) generating a laser beam of ultrashort pulses, wherein the pulses have a duration of about 100 fs to about 50 ps, a repetition rate of about 1 Hz to about 500 kHz, a pulse energy of about 1 to about 100 μJ, and a wavelength of between about 776 nm and 1552 nm; (iii) applying and focusing the beam to the surface of the biological tissue at a first site with a focus spot size in the range of 2-10 μm, wherein the beam is in a direction normal to the surface of the tissue and to a depth of the unwanted material, and wherein the focused beam induces optical breakdown and removes the unwanted material at the first site via laser-induced plasma ablation; and (iv) repeating the application of the focused beam to the surface of the biological tissue at a plurality of sites across the surface of the biological tissue, wherein: (a) the focused beam ablate the unwanted material at the plurality of sites, (b) the plurality of sites are adjacent to each other, and (c) the plurality of sites form an area, thereby resulting in ablation of material from an area of the surface of a biological tissue.

In other embodiments, the invention relates to a method of precision ablating unwanted material from an area on a surface of a biological tissue, comprising applying a USP laser to the surface of the tissue, wherein the laser does not induce damage to the tissue below the unwanted material. In certain embodiments, the method further comprises: (i) generating a laser beam of ultrashort pulses, wherein the pulses have a duration of about 100 fs to about 50 ps, a repetition rate of about 1 Hz to about 500 kHz, a pulse energy of about 1 to about 100 μJ, and a wavelength of between about 776 nm and 1552 nm; (ii) focusing the beam to the surface of the biological tissue at a first site with a focus spot size in the range of 2-10 μm, wherein the beam is in a direction normal to the surface of the tissue and to a depth of the unwanted material, and wherein the focused beam induces optical breakdown and removes the unwanted material at the first site via laser-induced plasma ablation; and (iii) repeating the application of the focused beam to the surface of the biological tissue at a plurality of sites across the surface of the biological tissue, wherein: (a) the focused beam ablate the unwanted material at the plurality of sites, (b) the plurality of sites are adjacent to each other, and (c) the plurality of sites form an area. In some embodiments, the USP laser is applied in a direction normal to the surface of the transverse laser. In other embodiments, the USP laser is applied in a direction parallel to the surface of the transverse layer. In certain embodiments, the method further comprises applying a diagnostic laser beam to the surface of the biological tissue to determine the depth of the unwanted material. In further embodiments, the depth to which the laser beam of ultrashort pulses is applied and the depth of the unwanted material determined by the diagnostic laser is essentially the same.

When ablating unwanted material from the surface of a biological tissue according to the methods of the subject invention, in certain embodiments, the ultrashort pulse laser beam passes through a non-biological material before contacting the surface of the biological tissue. Examples of non-biological materials include glass or a transparent or translucent plastic. In some embodiments, the transparent or translucent plastic encloses the biological tissue. In certain embodiments, the beam is channeled through the non-biological material via glass or plastic fibers.

In certain embodiments, ablation of unwanted material according to the methods described herein results in sterilization of the area of the surface of the biological tissue. In some embodiments, the area encompasses the entire surface of the biological tissue.

In some embodiments, the invention relates to a method of removing an internal volume from a material without damaging the surface of the material, comprising applying a USP laser to the material. In certain embodiments, the pulses of the laser have a duration of about 100 fs to about 50 ps, a repetition rate of about 1 Hz to about 500 kHz, a pulse energy of about 1 to about 100 μJ, and a wavelength of between about 776 nm and 1552 nm. In yet other embodiments, the method further comprises focusing the USP laser to the material at a first site where the internal volume is to be removed, wherein the focused laser induces optical breakdown and ablates at a depth of the internal volume, and repeating the application of the focused laser to the material at a plurality of sites across the material and to the depth of the internal volume, wherein the focused laser induces optical breakdown and ablates the internal volume. In certain embodiments, the method further comprises applying a diagnostic laser to the material to determine the depth of the internal volume. In some embodiments, the depth to which the laser beam of ultrashort pulses is applied and the depth of the internal volume determined by the diagnostic laser is essentially the same. In certain embodiments, the internal volume is a geometric shape or pattern. In certain embodiments, the material is a non-biological material. Examples of suitable non-biological materials include polymers, metals, and ceramics.

In some embodiments, the present invention relates to a method of removing an internal volume from a material without damaging the surface of the material, comprising: providing a material having an internal volume; generating a laser beam of ultrashort pulses, wherein the pulses of the laser have a duration of about 100 fs to about 50 ps, a repetition rate of about 1 Hz to about 500 kHz, a pulse energy of about 1 to about 100 μJ, and a wavelength of between about 776 nm and 1552 nm; applying and focusing the USP laser to the material at a first site where the internal volume is to be removed, wherein the focused laser induces optical breakdown and ablates at a depth of the internal volume; and repeating the application of the focused laser to the material at a plurality of sites across the material and to the depth of the internal volume, wherein the focused laser induces optical breakdown and ablates the internal volume, thereby removing the internal volume from the material.

In certain embodiments, the methods of the subject invention comprise applying a plurality of laser beams of ultrashort pulses to biological tissue.

### BRIEF DESCRIPTION OF THE DRAWINGS

FIGS. 1a and 1b show application of a USP laser to a biological tissue to separate a transverse layer from the tissue. FIG. 1a shows a schematic of application of the USP laser normal to the transverse layer of biological tissue, and FIG. 1b shows a schematic of application of the USP laser parallel to the transverse layer of biological tissue.

FIGS. 2a and 2b show the experimental set-up of a USP laser. FIG. 2a shows a schematic of the experimental set-up, and FIG. 2b shows a view of the work stage.

FIGS. 3a and 3b show porcine skin ablated by USP laser beam. FIG. 3a shows a macroscopic view of the porcine skin, wherein a black arrow identifies an ablated area. FIG. 3b shows a magnified view (10×) of the ablated porcine skin.

FIGS. 4a-4f show ablation of mold grown on a collagen gel by application of USP laser. FIG. 4a shows the collagen gel before ablation. FIG. 4b shows a magnified view of the surface of the collagen gel before ablation. FIGS. 4c and 4d show a magnified view of the surface of the collagen gel after ablation. The numbers mark individual “bands” of the ablated surface wherein the USP laser was applied across the surface. FIG. 4e shows a broader view of the collagen gel surface before ablation, and FIG. 4f shows the same view after ablation. Each “band” represents ablation induced by USP laser applied at a different working distance.

FIGS. 5a and 5b show ablation of blood smeared on glass. FIG. 5a shows the blood on the glass before application of the USP laser, and FIG. 5b shows the blood after application of the laser.

FIGS. 6a and 6b show ablation of beef blood smeared on a glass slide. FIG. 6a shows the blood smear ablated by USP laser applied at different repetition rates. FIG. 6b is a scanning electron microscopy (SEM) image at 3000× at the edge of an ablated region.

FIG. 7 shows ablation of sheep blood smeared on a glass slide by USP laser. Each line represents ablation by USP laser applied at different pulse energies.

FIGS. 8a and 8b show ablation of a layer of sheep red blood cells by USP laser. FIG. 8a shows the layer of red blood cells before ablation. FIG. 8b shows an area of the red blood cell layer ablated by USP laser, marked by a box.

FIGS. 9a and 9b show ablation of a layer of sheep red blood cells by USP laser. FIG. 9a shows an SEM image (1980×) of the layer of red blood cells before ablation. FIG. 9b shows an SEM image (2360×) of the layer of red blood cells after ablation by USP laser.

FIGS. 10a-10c show ablation of blood on a glass slide by USP laser applied through a packaging material. FIG. 10a shows the blood on the glass slide after ablation, wherein the slide is still covered by the packaging material. FIG. 10b shows the blood on the glass slide after ablation, and the slide packaging material removed from the glass. FIG. 10c shows the packaging material after rinsing with water.

FIGS. 11a and 11b show magnified views (40×) of the packaging material that covered the blood on a glass slide during ablation by USP laser, wherein the ejecta caused by the ablation adhered to the packaging material.

FIGS. 12a and 12b show ablation by USP laser of blood on a glass slide partially covered by a packaging material. FIG. 12a shows the blood on the glass slide after ablation, wherein the slide is still partially covered by the packaging material. FIG. 12b shows the blood on the glass slide after ablation, wherein the packaging material partially covering the slide is removed. The numbered bands in both FIGS. 12a and 12b represent ablation generated by USP laser applied at various working distances.

FIGS. 13a-13c show ablation by USP laser of blood smeared on a polydimethylsiloxane (PDMS) sample. FIG. 13a shows the smeared blood on the PDMS sample before ablation. FIG. 13b shows the smeared blood on the PDMS sample after ablation. FIG. 13c shows a magnified view of the portion of the ablated surface enclosed in a box in FIG. 13b.

FIGS. 14a and 14b show ablation by USP laser of blood smeared on tissue that is essentially flat. FIG. 14a shows tissue with smeared blood before application of the USP laser, while FIG. 14b shows the tissue after application of the laser.

FIGS. 15a and 15b show ablation by USP laser of blood smeared on tissue that has a curved surface. FIG. 15a shows tissue with smeared blood before application of the USP laser, while FIG. 15b shows the tissue after application of the laser.

FIGS. 16a and 16b show ablation by USP laser of LNCaP cells adhered to the surface of a slide. FIG. 16a shows the LNCaP cells before ablation, while FIG. 16b shows the LNCaP cells after ablation.

FIGS. 17a and 17b show ablation by USP laser of E. coli bacteria cultured on an agar plate and incubated for 12 hours. FIG. 17a shows the E. coli on the agar plate before ablation, and FIG. 17b shows the E. coli after ablation, wherein the ablated region is enclosed in the white box.

FIGS. 18a and 18b show ablation by USP laser of E. coli bacteria cultured on an agar plate and incubated for 36 hours. FIG. 18a shows the E. coli before ablation, while FIG. 18b shows the E. coli after ablation.

FIGS. 19a-19d show magnified lateral views of PDMS samples subjected to USP laser applied at different repetition rates to separate a transverse layer of the samples. FIGS. 19a-19d relate to USP laser applied at repetition rates of 500 kHz, 100 kHz, 20 kHz, and 5 kHz, respectively.

FIG. 20 shows a magnified lateral view of a PDMS sample subjected to USP laser applied at a repetition rate of 5 kHz and a pulse energy of 2 μJ to separate a transverse layer from the sample.

FIGS. 21a-21c show separation of a transverse layer of a PDMS sample by USP laser. FIG. 21a shows a top view of the PDMS sample on a glass slide before application of the laser. FIG. 21b shows a top view of the PDMS sample after application of the laser to separate a transverse layer. FIG. 21c shows a lateral view of the PDMS sample after application of the laser to separate a transverse layer, wherein forceps are used to show the separated layers.

FIGS. 22a and 22b show ablation by USP laser of material inside of a PDMS sample to create various shapes without disrupting the surrounding material. FIG. 22a shows V-shaped space created inside of a PDMS sample by ablation. FIG. 22b shows branching micro-channels generated inside of a PDMS sample by ablation.

FIGS. 23a and 23b show partial separation of a transverse layer of an epidermis sample by USP laser. FIG. 23a shows the epidermis sample before application of USP laser to separate a transverse layer, and FIG. 23b shows the sample after application of the USP laser.

FIGS. 24a and 24b depict (a) experimental setup I for single line ablation and (b) experimental setup II for multi-line ablation and separation.

FIG. 25 depicts a microscopic view of wet tissue ablation lines with different irradiation pulse energies.

FIGS. 26a, 26b, 26c, and 26d depict SEM images of the single line ablations with a fixed pulse overlap rate 20 pulses/μm and different irradiation energies: (a) 2.5 μK; (b) 2.0 μJ; (c) 1.5 μJ; and (d) 1.0 μJ.

FIG. 27 is a graph depicting square of ablation line width versus irradiation pulse energy for the evaluation of effective focal spot size.

FIG. 28 is a graph depicting single line ablation depths as a function of irradiation pulse energy.

FIG. 29 depicts histological views of single line ablation of wet tissue.

FIG. 30 depicts histological views of multi-line ablation of wet tissue.

FIGS. 31a and 31b depict wet tissue separation by USP laser ablation: (a) the dermis before laser ablation; and (b) the two separated thin layers.

FIG. 32 depicts an image of a partially separated dermis.

### DETAILED DESCRIPTION OF THE INVENTION

Described herein are methods and related compositions for separating a biological tissue into one or more layers or portions or removing unwanted material from the surface of a biological tissue using an ultrashort pulse (USP) laser.

### Ultrashort Pulse (USP) Laser

The term “ultrashort pulse laser” or “USP laser” refers to a laser beam generated in the form of extremely brief and finite intervals, i.e., pulses. USP lasers used herein are characterized by various parameters. For instance, “pulse duration” refers to the length of time of each interval wherein the laser beam is generated. A suitable pulse duration may be, e.g., between about 100 fs to about 50 ps, preferably between about 500 fs to about 10 ps, more preferably between about 1 ps to about 5 ps.

The parameter “pulse energy” refers to the amount of energy concentrated in each interval wherein the laser beam is generated. Pulse energy may be between about 0.5 μJ to about 100 μJ, more preferably between about 1 μJ to about 5 μJ.

The parameter “repetition rate” refers to the number of pulses that are emitted per second, and indirectly relates to the time between each pulse emission, i.e., the length of time between each pulse. The repetition rate may be between about 1 Hz and about 100 MHz, preferably between about 100 Hz and about 500 kHz, more preferably between about 1 kHz and about 100 kHz.

Another parameter used to characterize the USP laser is “scanning velocity,” which refers to the rate at which the USP laser moves across the surface of a material. The scanning velocity may be, for example, between about 1 mm/s and about 50 mm/s, preferably between about 5 mm/s and about 20 mm/s. Alternatively, the scanning velocity can be expressed as “pulses/μm.” Described in units, scanning velocity may be between about 0.1 pulses/mm and about 10 pulses/μm, preferably between about 0.5 pulses/μm and about 5 pulses/μm, more preferably between about 1 pulse/μm and about 3 pulses/μm.

The “scanning line width” or “focus spot size” refers to the diameter of the USP laser beam. This diameter may be, for example, between about 1 μm and about 20 μm, preferably between about 2 μm and about 10 μm, and more preferably between about 3 μm and about 5 μm.

The USP laser beam of the invention may be of any wavelength in the electromagnetic spectrum, but is preferably about 1552 nm.

The methods of the invention described herein take advantage of the unique effects of USP lasers. Specifically, USP lasers can remove material from a target site via plasma-induced ablation. Plasma-induced ablation involves the application of a laser at an intensity that is above the optical breakdown threshold, i.e., about 10¹¹ W/cm². This causes a strong local ionization at the target site, where the plasma reaches densities beyond the critical value of between 10²⁰ and 10²² electrons/cm³. The laser energy is efficiently absorbed by the plasma, and the local plasma temperature increases.

If the USP laser power is high, this can result in an explosive Coulombian expansion that produces cavitation. These cavities can collapse, and any small amount of gas within the cavities will dissipate rapidly, producing a powerful and even damaging shockwave. If the pulse rate of the laser is slow, energy is transferred from the plasma to the lattice, and thermal damages can occur.

Advantageously, the USP laser of the present invention is applied at a laser intensity of about 0.5 μJ to about 10 μJ, a wavelength of 1552 nm, and a pulse duration of about 100 fs to about 50 ps. Consequently, this minimizes the effects of cavitation and the transfer of energy to the lattice. The ablated material at the target site is thereby converted to plasmas without thermal damage to the surrounding material. This mechanism occurs whether the USP laser is focused to a depth within a material, or to the surface of the material. Therefore, USP lasers serve as an ideal instrument for processing allograft, xenograft, and autograft tissues due to their ability to ablate material at a target site without damage to surrounding material.

The property of USP lasers of the invention to ablate material from a target site without transferring energy and damaging surrounding material is ideal for precision methods, e.g., methods relating to precision separation, precision cutting, precision ablation, etc. The term “precision” relates to application of the USP laser wherein little, if any, damage results to material surrounding the target site. Because of the very short interaction time, thermal damage to surrounding medium is minimized. Accordingly, in these embodiments, precision application of the USP laser will generally result in a clean and well-defined removal of target material.

### Biological Tissues

The term “biological tissue” or “biological material” used herein includes any material derived from a living or once-living source. Importantly, these include allograft, xenograft, and autograft tissues (collectively referred to herein as “grafts”), as well as biologic matrices derived from tissue sources.

The term “allograft” refers to a transplant comprising cells, tissues, or organs sourced from another member of the same species. The member of the same species may be living or nonliving.

The term “xenograft” refers to a transplant comprising cells, tissues, or organs sourced from another species. Examples of species that commonly serve as a xenograft source include, but are not limited to, simian, porcine, bovine, ovine, equine, feline, and canine.

Finally, the term “autograft” refers to cells, tissues, or organs transplanted from one site to another on the same patient.

Examples of tissues that are typically used as an allograft, xenograft, or autograft include, but are not limited to, musculoskeletal tissues such as bone grafts, and muscle; cardiovascular tissue such as heart valves and blood vessels, connective tissue such as ligaments, tendons, and cartilage; dermal tissue such as dermis, epidermis, and whole skin; and neural tissue.

Alternatively, the biological tissue may be a biologic matrix derived from any number of tissue sources, in particular soft tissue sources, including dermal, fascia, dura, pericardia, tendons, ligaments, and muscle.

Example of biologic matrices suitable for the present invention are set forth in U.S. Provisional Application Ser. No. 61/030,930, filed Feb. 22, 2008 and International Application No. PCT/US09/34891, filed February 23, 2009, which are each incorporated herein by reference in their entirety. Suitable dermal matrices include, for example, acellular dermal matrices such as the human acellular dermal matrices from the Flex HD® product line (available from Musculoskeletal Transplant Foundation, Edison, N.J.).

Biologic matrices are suitable for use in surgical procedures for the replacement of damaged or inadequate integumental tissue or for the repair, reinforcement or supplemental support of soft tissue defects, such as ventral or abdominal hernia, and abdominal wall repair; breast reconstruction; cranial, maxillary, facial reconstruction; urologic and gynecologic reconstructions; bladder neck suspensions; rotator cuff and other tendon repair; chronic and acute wound care; burn care; dura repair and replacement; gastrointestinal reconstructions; parastomal reinforcement and repair; trauma repairs; and diabetic ulcers and chronic venous insufficiency ulcers.

The term “biological tissue” or “biological material” may also refer to bone, muscle, fascia, bladder, stomach, heart, small intestine, large intestine, and parenchymal organs such as the liver, pancreas, lungs, etc.

The term “ablation” or “ablate” refers to removal of material. This includes removal of material by melting or vaporization.

Application of USP Laser to Separate Biological Tissue into Layers

One particular aspect of the invention provides a method of separating a transverse layer from a biological tissue without damaging the surface of the transverse layer, comprising applying a USP laser beam to the biological tissue. The beam may be initially focused at a depth below the transverse layer at a first site, such that the beam ablates the biological material at the site. The USP laser may then be applied to a second site below the transverse layer and adjacent to the first site, wherein the laser ablates material at the second site. This process may be repeated for additional sites across the biological tissue below the depth of the transverse layer until all the material connecting the transverse layer with the bulk biological tissue has been removed. This allows the transverse layer to separate from the biological tissue.

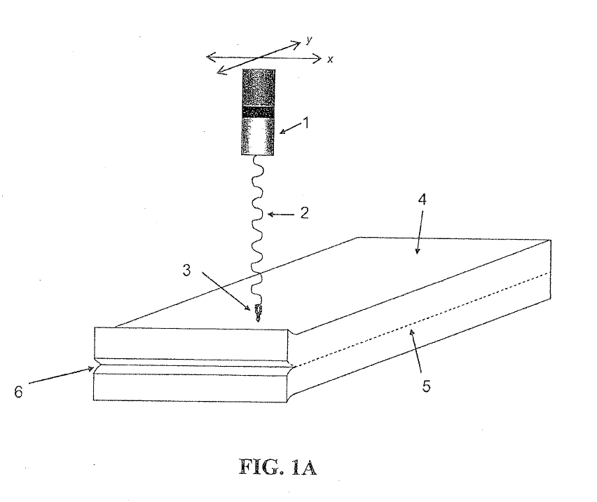

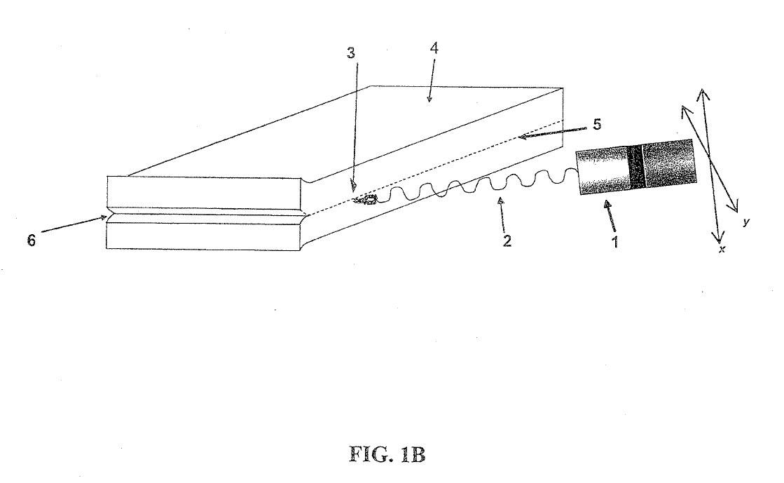

FIGS. 1a and 1b show the application of a USP laser to a biological tissue to separate a transverse layer. The laser source 1 applies the USP laser 2 to the biological tissue 4. The beam penetrates 3 the biological tissue 4 and focuses to a depth below the transverse layer 5 that is to be separated. The USP laser induces ablation of the biological tissue 4 at a depth to produce a separation 6 of the layer.

Another particular aspect of the invention is a method of precision separating a transverse layer from a biological tissue without damaging the surface of the transverse layer and without damaging the tissue surrounding the separated layer, comprising applying a USP laser beam to the biological tissue. This method takes advantage of the USP laser beam's capability to ablate material without transferring energy to the surrounding material. In this method, the beam may be focused at a depth below the transverse layer at a first site, such that the beam ablates the biological material at the site without damaging or affecting the surrounding material. The USP laser may then be applied to a second site below the transverse layer and adjacent to the first site, wherein the laser ablates material at the second site without damaging the surrounding material. This process may be repeated for additional sites across the biological tissue below the depth of the transverse layer until the all material connecting the transverse layer with the bulk biological tissue has been removed. This allows the transverse layer to separate from the biological tissue.

In a preferred embodiment, the USP beam is applied in a direction that is normal to the surface of the biological material. In an alternative embodiment, the beam is applied in a direction parallel to the transverse layer.

As used herein, “donor site” or “donor area” refers to the area wherein the graft, e.g., allograft, xenograft, or autograft, is excised. “Receiving site” or “receiving area” refers to the area of the patient to which the graft will be implanted.

In certain embodiments, the USP laser is used to separate layers of biological materials such as allografts, xenografts, autografts, and biologic matrices. As described above, the biological materials may be musculoskeletal, cardiovascular, connective, neural, or dermal.

In particular embodiments, the biological material is dermal.

In certain embodiments, the USP laser is used to excise a dermal graft from a donor site. In other embodiments, the USP laser can be used to prepare full-thickness skin grafts (FTSG), which comprise the complete epidermis and dermis. At the donor site of the biological material, the USP laser can be applied and focused to a depth below the epidermal layer to ablate biological material at that depth. This process is repeated throughout the graft area of skin intended to be excised. The USP laser is also applied at the edges of the graft area for the full thickness of the graft in order to separate the sides of the graft from the surrounding material. The separation of the sides of the graft from the surrounding material and the separation of the bottom of the graft from the underlying material can occur in no particular order, and these steps may be combined or mixed. Once completed, the resulting graft can be removed from the remaining material.

In one particular embodiment, the USP laser may be applied at a depth which includes superficial fat. Once excised, the fat may be removed by scissors and the like, or by USP laser which may be applied in a direction parallel to the dermal and epidermal skin layers.

The USP laser can excise FTSG from essentially all sites throughout the body including, but not limited to, preauricular, postauricular, supraclavicular, and clavicular areas, as well as the neck, nasolabial folds, and eyelids. The selection criteria for the area wherein the graft will be excised are known in the art, but include matching skin texture, thickness, color, and actinic damage between the donor site and the receiving site.

In another embodiment, a portion of the skin is already excised from surrounding tissue, and USP laser is applied to only separate the dermal layer from underlying tissue. In this case, the sample may have been excised by the USP laser as described above, or by another means known in the art, e.g., dermatome, a Week blade, etc.

In certain embodiments, the USP laser can be used to prepare split-thickness skin grafts (STSG), which comprise the complete epidermis and part of the dermis. In the preparation of STSG, the USP laser can be applied and focused to a depth within the dermal layer at the donor site to ablate biological material at that depth. This process is repeated throughout the area of skin intended to be excised. The USP laser is likewise applied at the edges of the graft area for the full thickness of the graft in order to separate the sides of the graft from the surrounding material. The separation of the sides of the graft and the separation of the bottom of the graft can occur in no particular order, and the steps may be combined or mixed. Once completed, the resulting graft can be removed from the remaining material.

The USP laser can excise STSG of various thicknesses, including grafts categorized in the art as Thiersch-Ollier grafts (0.15-0.3 mm), Blair-Brown grafts (0.3-0.45 mm), and Padgett grafts (0.45-0.6 mm). Alternatively, the thickness may encompass the epidermal layer only. The selection criteria of the thickness of the graft are known in the art, but includes considering the receiving site's requirements for durability, cosmetics, and healing time.

The USP laser can excise STSG from essentially all donor sites on the body. The selection criteria for the donor site is known in the art, but includes the patient's ability to ambulate, sit, and sleep. Examples of donor sites include, but are not limited to, abdomen, buttock, inner and outer arm, inner forearm and thigh.

In another embodiment, the laser skin sample is already excised from surrounding tissue, and the laser may be applied to only separate the epidermal layer and part of the dermal layer from underlying tissue, or even separate the epidermis from the dermis. The sample may have been excised by the USP laser as described above, or by another means known in the art, e.g., dermatome, a Weck blade, etc.

In other embodiments, the USP laser can be used to prepare skin flaps. A skin flap is a full-thickness portion of the skin, including the subcutaneous fat, which is sectioned and separated from the surrounding skin except on one side, which is called the peduncle. Skin flaps are typically advanced or rotated laterally in order to cover nearby losses of skin. The skin flap may be formed by applying the USP laser to the skin and focusing the laser to a depth within or immediately below the subcutaneous fat. This process is repeated throughout the flap area of skin intended to be separated. The USP laser is also applied at the edges of the flap area for the full thickness except for the peduncle. The separation of the sides of the flap from the surrounding tissue and the separation of the bottom of the flap from the underlying tissue can occur in no particular order, and the steps may be combined or mixed.

The USP laser can prepare skin flaps from essentially all donor sites on the body. The size and shape of the skin flap may vary according to repair needs, including the site of the repair. Repairs involving skin flaps initially rely on the blood supply provided through the peduncle, and therefore skin flaps for repairs at sites with high vascularity can have a higher length:width ratio than skins flaps for repairs at sites with low vascularity. For instance, skin flaps prepared for repairs on the face can have a length/width ratio of about 3:1 to 4:1, while flaps prepared for repairs on the trunk and limbs are typically below a length/width ratio of about 2:1.

The general protocol for preparing the donor area and removing the graft is also well known in the art. For example, the procedure may include removing the area of all hair to aid in the harvesting and handling of the graft. Hair can be removed by methods known in the art such as with a razor or hair-removal chemicals, but may also be removed by application of USP laser (see below). Local anesthesia is typically applied, although, depending on the site of the graft to be harvested, regional anesthesia may be applied as an alternative or in combination. The donor site area may be scrubbed and prepared with a surgical antiseptic or cleanser such as, for example, povidone-iodine and chlorhexidine gluconate. All antiseptic residues may be washed off with a sterile saline and the donor area may be dried. The site may be marked with a surgical marking pen or the like. Optionally, a semipermeable membrane may be placed over the donor site to minimize contraction and curling of the graft after application of the USP laser. The skin may be pulled tight, and the USP laser is applied. After the graft has been separated from the surrounding tissue, the graft can be elevated using means known in the art, such as forceps, a skin hook, a needle tip, or suction.

Advantageously, accurate and careful separation of the epidermis from the dermis without damaging either skin layer increases the efficiency of the sample, and allows for either layer to be used in separate applications, e.g., epidermis for treating skin wounds, dermis for preparing biologic matrices.

In other embodiments, the biological tissue may be bone, muscle, fascia, bladder, stomach, heart, small intestine, large intestine, and parenchymal organs.

Another embodiment relates to a method of removing an internal volume from a biological tissue and creating a cavity within the tissue without damaging or affecting the surface or creating an opening to create the cavity, comprising applying a USP laser to the biological tissue. Cavities may be formed for a variety of reasons, such as to remove diseased tissue or to prepare the material for implant fixation. The USP laser can be applied and focused at an initial site where the internal volume is to be removed at a depth below the surface of the tissue to ablate biological material at that depth. This process is repeated at sites adjacent to the initial site until the cavity is created. The cavity can be of varying size and shape, e.g., holes, geometric shapes, microchannels, and can be applied to various biological tissues as described above. This process can also be applied to non-biological materials, such as polymers, metals, and ceramics.

### Application of USP Laser to Cut Biological Tissue

Another aspect of the invention relates to a method of cutting a biological tissue comprising applying a USP laser beam to the biological tissue. The beam may be focused on the biological tissue at a first site where the cut is to occur in order to induce ablation of material at the first site. The beam may then be focused on a second site of the material adjacent to the first site, but also where the cut is to occur, in order to ablate material at the second site. This process may be repeated through the depth of the biological material, or across the length/width of the biological material until the desired cut is formed. The USP laser beam can be used to cut the tissue into one or more separate portions. In certain embodiments, the USP laser may be used to cut the biological tissue into a desired shape or form.

Another aspect of the invention is a method of precision cutting of a biological tissue without damaging the tissue surrounding the cut, comprising applying a USP laser beam to the biological tissue. The USP laser beam ablates material at the cut without transferring energy to the surrounding tissue which could lead to damage. In this method, the beam may be on the biological tissue at a first site where the cut is to occur in order to ablate material at the site without damaging the surrounding tissue. The beam may then be focused at a second site where the cut is to occur in order to ablate material without damaging surrounding tissue at the second site. This process can be repeated until the desired cut is formed. The cut may separate the tissue into two or more portions, and these portions may be of any desired shape.

The beam may be applied in a direction normal to the surface of the biological tissue, or parallel to the surface of the biological tissue.

In a preferred embodiment, the biological tissue may be an excised allograft, xenograft, autograft, or biologic matrix. The biological tissue may be bone, muscle, fascia, bladder, stomach, heart, small intestine, large intestine, and parenchymal organs, as described above.

Application of USP Laser to Remove Unwanted Materials from Biological Tissue

A further aspect of the invention is a method of ablating unwanted material from an area on a surface of a biological tissue comprising applying a USP laser beam onto the tissue surface. The beam can be initially focused on the unwanted material at a first site, wherein the beam induces ablation of the unwanted material at the site. The USP laser can then be applied to a second site adjacent to the first site, such that the USP laser will ablate the unwanted material at the second site. This process can be repeated for additional sites until the unwanted material is ablated from the desired area on the surface of the biological tissue.

Another aspect of the invention is a method of precision ablating of unwanted material from an area on a surface of a biological tissue comprising applying a USP laser beam onto the tissue surface without damaging the tissue beneath the unwanted material. The beam can be initially focused on the unwanted material at a first site, wherein the beam induces ablation of the unwanted material at the site without damaging the tissue below. The USP laser can then be applied to a second site adjacent to the first site, such that the USP laser will ablate the unwanted material at the second site without damaging the tissue below the second site. This process can be repeated for additional sites until the unwanted material is ablated from the desired area on the surface of the biological tissue.

The beam may be applied in a direction normal to the surface of the biological tissue, or parallel to the surface of the biological tissue. In one embodiment, the USP laser will be used to remove unwanted material from the entire surface of the biological tissue.

The unwanted materials removed from the surface of the biological material may be contaminants that compromise the safety or sterility of the tissue. Such contaminants include gram positive bacteria, gram negative bacteria, spore-forming bacteria, yeasts, and fungi. Examples of gram positive bacteria are Clostridium spp, Aerococcus, Micrococcus, Staphylococcus aureus, Staphylococcus sciuri, Staphylococcus epidermidis, and Bacillus cereus. Examples of gram negative bacteria are Acinetobacter and E coli.

The application of a USP laser to remove contaminants can be used in combination with other methods of disinfecting and sterilizing biological material, such as the aseptic processing technology practiced by the Musculoskeletal Transplant Foundation in the production of Flex HD®, DermaMatrix®, and Epliflex®.

The unwanted materials may also comprise a layer of cells. This includes, for example, removal of the periosteum for bone grafts, or the removal of viable cells in skin grafts.

In the case wherein the biological tissue is dermal, the unwanted material may be hair. The unwanted material may further comprise the hair shaft if the dermal tissue is from a non-living source, or may further comprise hair follicles if the dermal tissue is from a living source.

In one embodiment of the invention, the USP laser passes through a second material before interacting with the biological tissue to remove the unwanted material from the surface. This second material may be glass or a transparent or translucent plastic used for packaging the biological tissue. Examples of packaging materials include TYVEK, which is a brand of flashspun high-density polyethylene (HDPE) fibers, and KAPAK polyester bags. During application of the USP laser, the beam focuses on a depth inside of the packaging material to remove unwanted materials from the surface of the biological tissue without damaging or disturbing the integrity of the packaging. This is especially useful when the biological tissue has been disinfected or sterilized before it was placed in the packaging material, and may be considered as a final step.

Optionally, the USP laser can be applied to biological material in packaging from a source outside of the aseptic processing area. For instance, the USP laser may be transmitted through glass into a separate sterilized room, and through packaging material to focus on biological material. Alternatively, the laser beam may be channeled into a room from another room via glass or plastic fibers. The fibers employ fiber optic technology known in the field, and transmits the laser beam from the laser source to the biological material. For example, the fibers may be single mode or multimode fibers, depending on the power of the beam and the distance that the beam must travel (see U.S. Pat. No. 4,785,806, which is incorporated herein by reference).

### Diagnostic Laser

In all of the embodiments provided above, a diagnostic laser may be used to determine the depth at which the USP laser beam should be applied. In a preferred embodiment, the diagnostic laser may determine the depth of the transverse layer to be separated from the biological tissue. In another preferred embodiment, the diagnostic laser may determine the depth of the unwanted material on the surface of the biological tissue.

The following non-limiting examples further describe and enable one of ordinary skill in the art to make and use the present invention.

### EXAMPLES

### Example 1

### Experimental Set-Up

A schematic of the experimental setup is shown in FIGS. 2a and 2b. An Erbium Doped Fiber Laser (Raydiance, Inc) operates at wavelength 1552 nm with a pulse duration of 1.1 pico-second was used in the experiments. The repetition rate is tunable between 1 to 500 KHz and the pulse energy is variable between 1-5 μJ. The laser beam generated by the system was modified by an astigmatism correction mirror and was launched into a long working distance objective lens (M Plan Apo NIR 20x/0.4 N.A., Mitutoyo). The energy loss after the lens is about 50-60%. The output focused beam has a diameter of about 8 μm.

The target sample was fixed to a lab-made attitude adjustable work fixture which was placed on a programmable 3-D automated Precision Compact Linear Stage (VP-25XA, Newport). The automated stage moves at a speed range between 1-25 mm/s.

In an alternative set-up, the stage can remain stationary while the laser source is mobile, or both the stage and the laser source may be mobile.

### Example 2

### Ablation of Porcine Skin by USP Laser

USP laser beam was applied to the surface of porcine skin to determine the skin's response. The beam was applied at the parameters shown in Table 1.

TABLE 1
Parameters of USP laser used
for ablating porcine skin.

| Pulse Parameter | Setting | |
|---|---|---|
| Duration | 1.1 | ps |
| Energy | 5 | μJ |
| Repetition Rate | 5.05 | kHz |
| Scanning Velocity | 5 | mm/s |
| Wavelength | 1552 | nm |

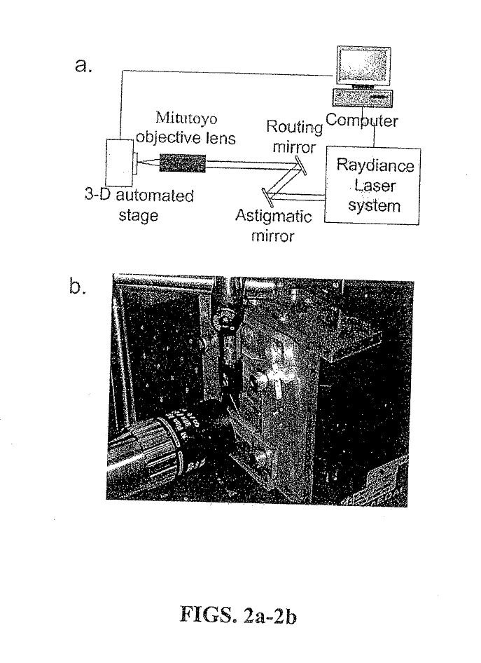

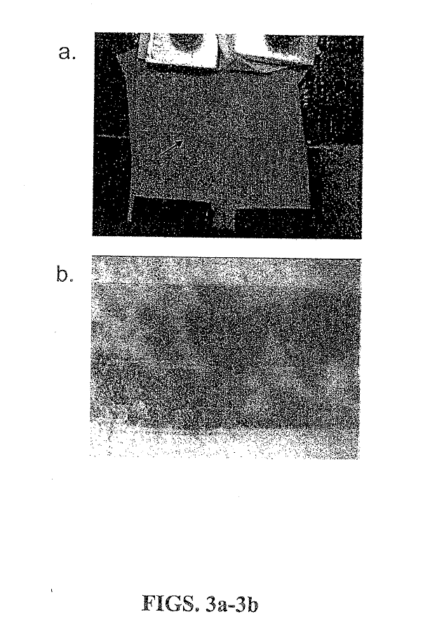

The porcine skin was adhered to a surface using attachments as shown in FIG. 3a. The beam was applied across the width of the skin sample in a direction normal to the skin surface. The skin displayed a distinct band wherein the surface of the skin has been ablated (see FIG. 3b),

### Example 2

### Ablation of Growth Media by USP Laser

USP laser beam was applied to a collagen gel having mold growth on its surface to determine whether the beam can remove the mold from the collagen gel surface. The beam was applied at the parameters shown in Table 2.

TABLE 2
Parameters of USP laser used for
ablating mold on a collagen gel.

| Pulse Parameter | Setting | |
|---|---|---|
| Duration | 1.1 | ps |
| Energy | 5 | μJ |
| Repetition Rate | 5.05 | kHz |
| Scanning Velocity | 5 | mm/s |
| Wavelength | 1552 | nm |

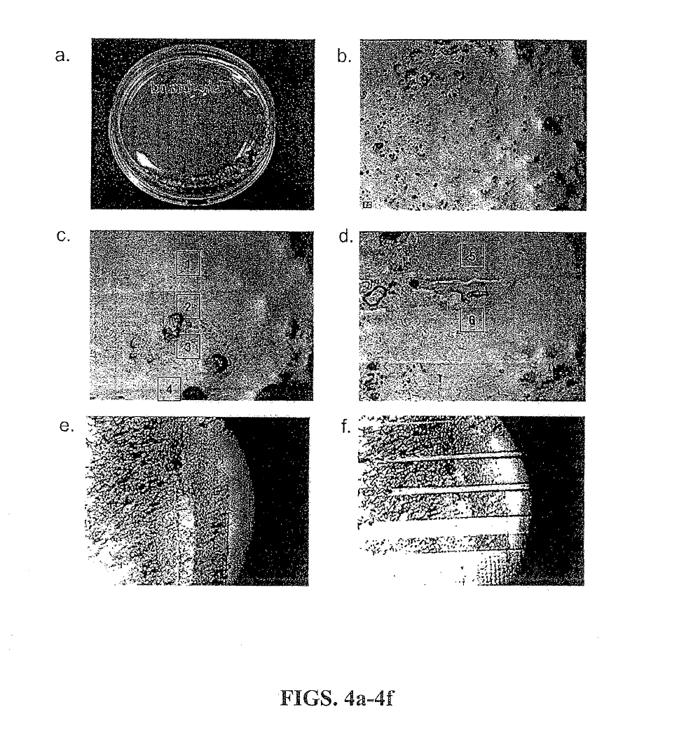

Mold was grown on the surface of a collagen gel, as shown in FIG. 4a. A magnified view of the surface of the collagen gel clearly shows that the mold grew across the surface of the gel (FIG. 4b).

The USP laser was applied at various working distances, i.e., the distances between the laser source and the sample. The effects of the USP laser on mold ablation are exhibited in FIGS. 4c and 4d, which show bands where the surface was ablated. The bands were generated by laser applied at different working distances, and suggest that the working distance influences the extent of ablation. For example, band #2 shows complete ablation of the mold from the collagen gel surface, while band #3 shows little, if any, ablation.

The effects of working distance are also demonstrated in FIGS. 4e and 4f. FIG. 4e shows a region of the collagen gel before ablation, while FIG. 4f shows the same region after application of the USP laser at various working distances, which results in bands of varying widths. These results suggest that working distance can also affect the scanning width of the laser.

Example 3: Ablation of Blood on a Glass Slide by USP Laser

USP laser beam was applied to a glass slide having blood on its surface to further demonstrate the capability of USP laser to remove unwanted material from a surface. The beam was applied at the parameters shown in Table 3.

TABLE 3
Parameters of USP laser used for
ablating blood from a glass slide.

| Pulse Parameter | Setting | |
|---|---|---|
| Duration | 1.1 | ps |
| Energy | 5 | μJ |
| Repetition Rate | 20 | kHz |
| Scanning Velocity | 20 | mm/s |
| Wavelength | 1552 | nm |

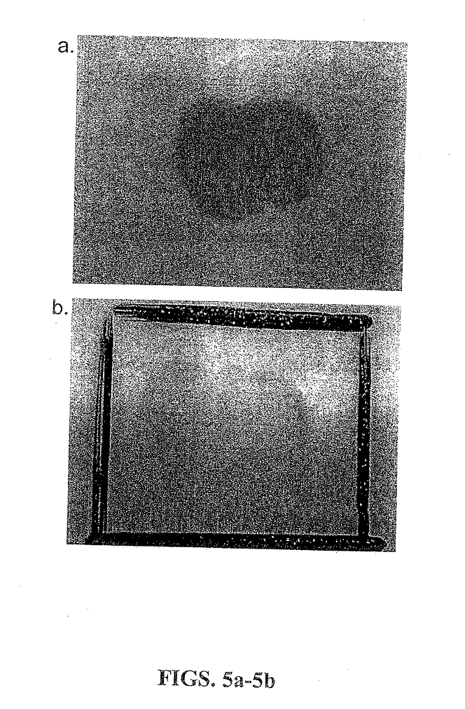

Application of the USP laser removed blood from the surface of glass, as shown in FIGS. 5a and 5b.

The relationship between the working distance and ablation depth was determined for both glass contaminated with blood and bare glass. Ablation depth was measured using DEKTAK 3030 Profilometer. The results are shown in Table 4.

TABLE 4
Relationship between working distance and ablation depth for
glass slide with and without contamination of blood.

| | Working Distance | Ablation Depth |
|---|---|---|
| Glass slide | (mm) | (μm) |
| Contaminated with Blood | 20.104 | 4.8 |
| Contaminated with Blood | 20.091 | 7.2 |
| Contaminated with Blood | 20.079 | 8.5 |
| Bare | 20.208 | 2.4 |
| Bare | 20.203 | 3.3 |
| Bare | 20.198 | 4.0 |

The effect of repetition rate of the USP laser on ablation depth was also determined. The USP laser beam was applied to a glass slide contaminated with beef blood at five different repetition rates. The effects of the repetition rates on the ablation depth are shown in Table 5.

TABLE 5
Relationship between repetition rates and ablation
depth of the USP laser beam.

| | Strip 1 | Strip 2 | Strip 3 | Strip 4 | Strip 5 |
|---|---|---|---|---|---|
| Repetition | 100 | 20 | 10 | 5.05 | 1.01 |
| Rate (kHz) | | | | | |
| Ablation | 2.4 ± 0.4 | 2.3 ± 0.2 | 3.0 ± 0.3 | 3.2 ± 0.5 | 0.5 ± 0.5 |
| Depth (μm) | | | | | |

Table 5 indicates that there is a non-linear relationship between repetition rate and ablation depth. The greatest ablation depth occurred at repetition rates of 10 kHz and 5.05 kHz, while both higher and lower repetition rates decreased the ablation depth.

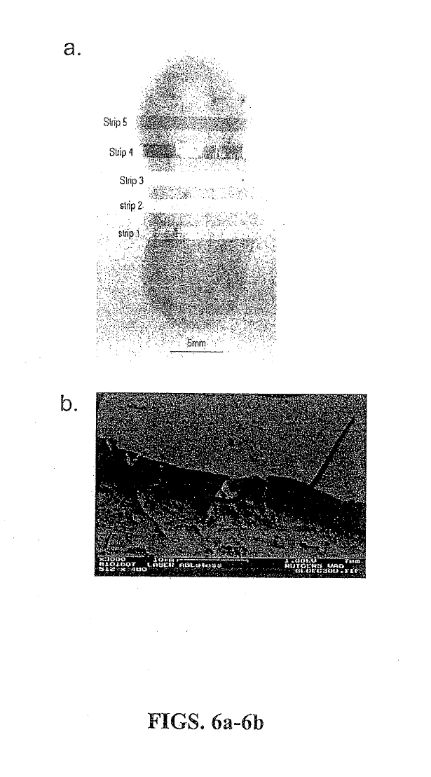

The effects of ablation depth can be seen in FIG. 6a, wherein strips 3 and 4, which have the greatest ablation depth, show nearly complete removal of blood by the USP laser. A magnified view of the strip shows how the blood has been removed (see FIG. 6b).

The USP laser beam was also applied to a glass slide contaminated with sheeps's blood at four different pulse energies to determine the effect of pulse energy on scanning line width. The scanning line width associated with various pulse energies are shown in Table 6.

TABLE 6
Relationship between pulse energies and scanning
line width of the USP laser beam.

| | Line 1 | Line 2 | Line 3 | Line 4 |
|---|---|---|---|---|
| Pulse Energy (μJ) | 5 | 4 | 3 | 2 |
| Scanning Line | 10.6 ± 4.1 | 9.6 ± 3.4 | 7.4 ± 2.2 | 6.4 ± 2.0 |
| Width (μm) | | | | |

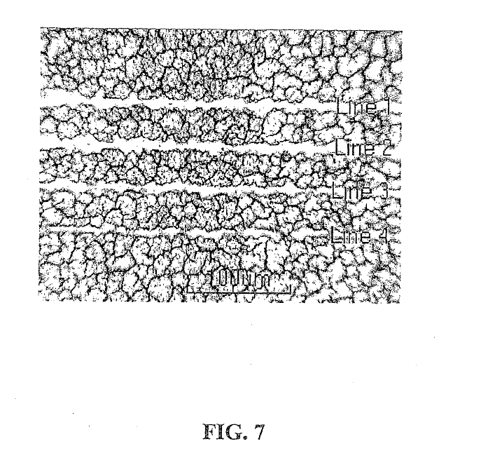

Table 6 indicates that, in general, application of the USP laser at higher pulse energies results in greater scanning line width. This is shown in FIG. 7.

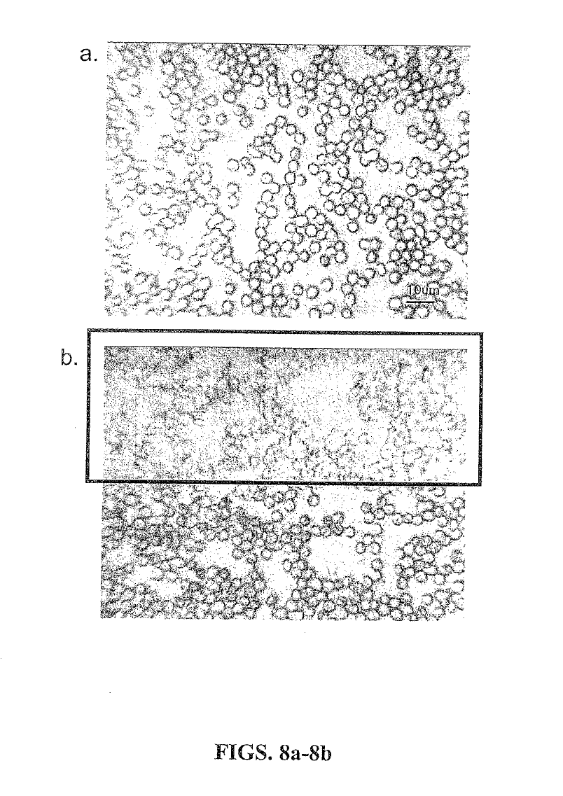

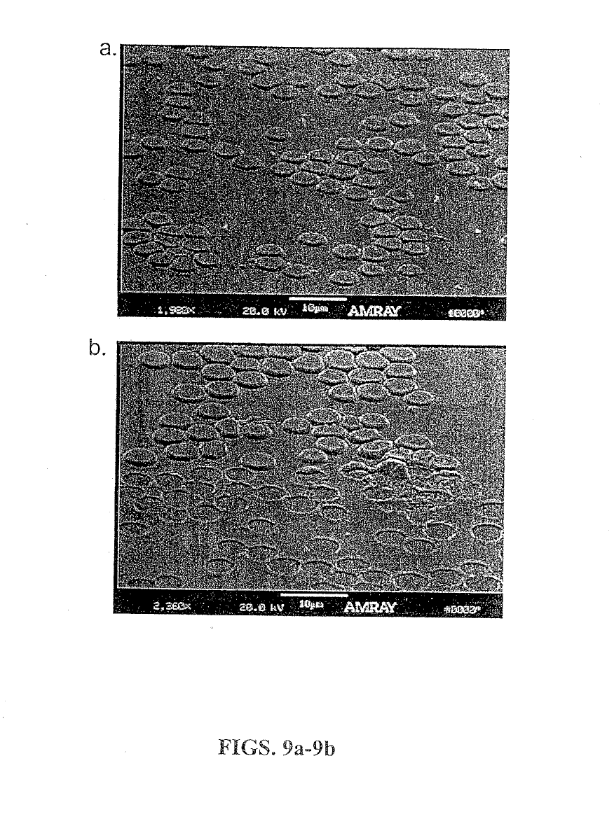

The effect of USP laser on red blood cells of sheep was also assessed. A magnified view of the slide before application of the USP laser shows a dense population of blood cells (FIGS. 8a, 9a), while a view of the slide after application of the shows a band of ablated cells where the laser was applied (FIGS. 8b, 9b).

### Example 4

### Ablation of Blood from Slide Covered with a Packaging Material by USP Laser

USP laser beam was applied to a slide having blood on its surface, such that the slide is covered with a translucent packaging material, in order to demonstrate the capability of the USP laser to ablate a surface through another material. The beam was applied at the parameters shown in Table 7.

TABLE 7
Parameters of USP laser used for ablating blood from a slide.

| Pulse Parameter | Setting | |
|---|---|---|
| Duration | 1.1 | ps |
| Energy | 5 | μJ |
| Repetition Rate | 5.05 | kHz |
| Scanning Velocity | 5 | mm/s |
| Wavelength | 1552 | nm |

A transmission test of the packaging materials (TYVEK and KAPAK) revealed how the beam was transmitted through the packaging. This is shown in Table 8.

TABLE 8
Transmission test of package materials.

| Package Component | Transmission at wavelength 1552 nm |
|---|---|
| Second layer | 89.6% |
| First layer, plastic side | 89.8% |
| First layer, fiber side | 15.9% |

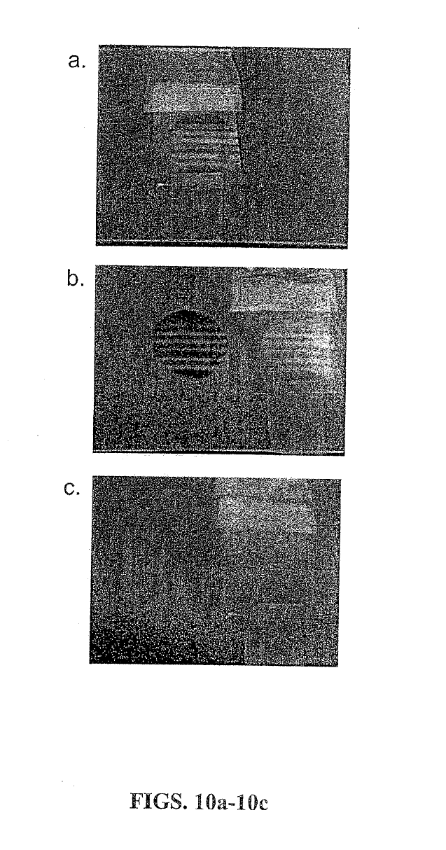

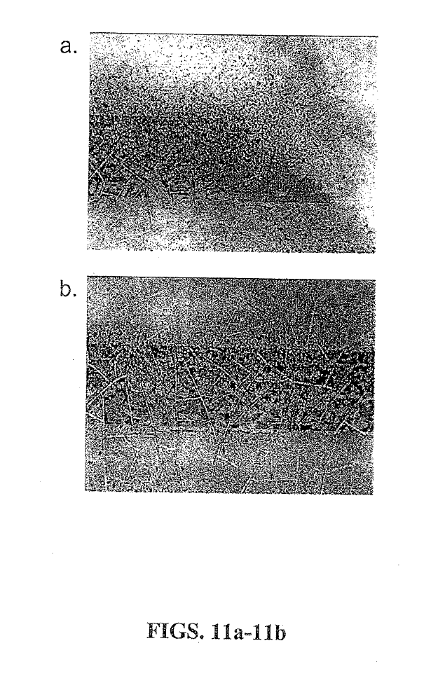

The USP laser beam ablated the blood from the surface of the slide through the packaging material. While the slide was still covered, bands identifying where the blood was ablated were visible (see FIG. 10a). Removal of the packaging material revealed that blood was indeed ablated across the sample (see FIG. 10b). The packaging material still had bands, which were the ejecta that were ablated from the slide. The packaging material was then washed, which removed the bands and confirmed that the bands on the packaging was indeed ejecta.

Magnified views of the ejecta on the surface of the packaging material are shown in FIGS. 11a and 11b.

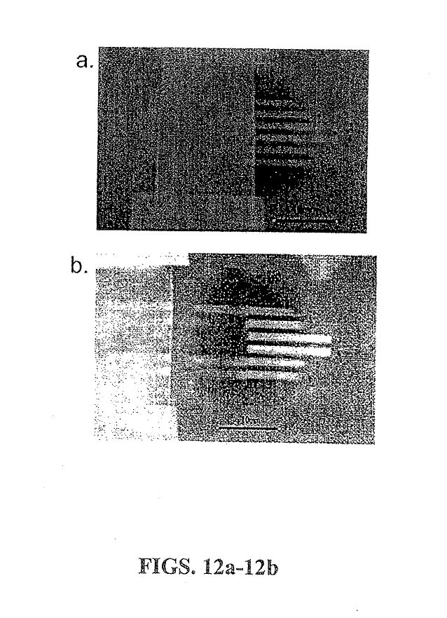

The capability of the USP laser to pass through a packaging material may be influenced by the working distance of the laser. As shown in FIGS. 12a and 12b, the packaging material prevented ablation of the blood when the USP laser was applied at certain working distances. This demonstrates that certain working distances are more effective for ablation through a transparent material.

### Example 5

### Ablation of Blood Contamination on PDMS Surface by USP Laser

USP laser beam was applied to a polydimethylsiloxane (PDMS) sample contaminated with beef blood plasma. The beam was applied at the parameters shown in Table 9.

TABLE 9
Parameters of USP laser used for ablating
blood from a PDMS sample.

| Pulse Parameter | Setting | |
|---|---|---|
| Duration | 1.1 | ps |
| Energy | 5 | μJ |
| Repetition Rate | 20 | kHz |
| Scanning Velocity | 20 | mm/s |
| Wavelength | 1552 | nm |

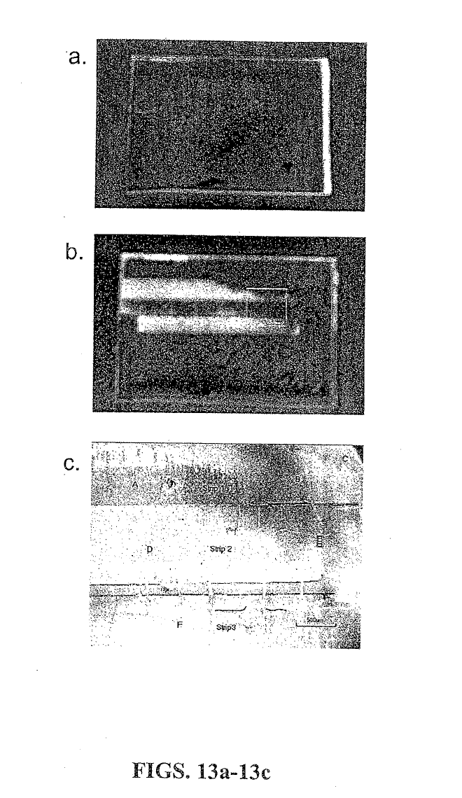

Beef blood was smeared onto the surface of the PDMS sample, as shown in FIG. 13a. Application of the USP laser created three distinct strips, which marks where the beef blood was ablated from the PDMS surface (see FIGS. 13b and 13c).

### Example 6

### Ablation of Blood Contamination on Tissue

USP laser beam was applied to a tissue sample contaminated with blood. The beam was applied at the parameters shown in Table 10.

TABLE 10
Parameters of USP laser used for ablating blood from tissue.

| Pulse Parameter | Setting | |
|---|---|---|
| Duration | 1.1 | ps |
| Energy | 5 | μJ |
| Repetition Rate | 20 | kHz |
| Scanning Velocity | 20 | mm/s |
| Wavelength | 1552 | nm |

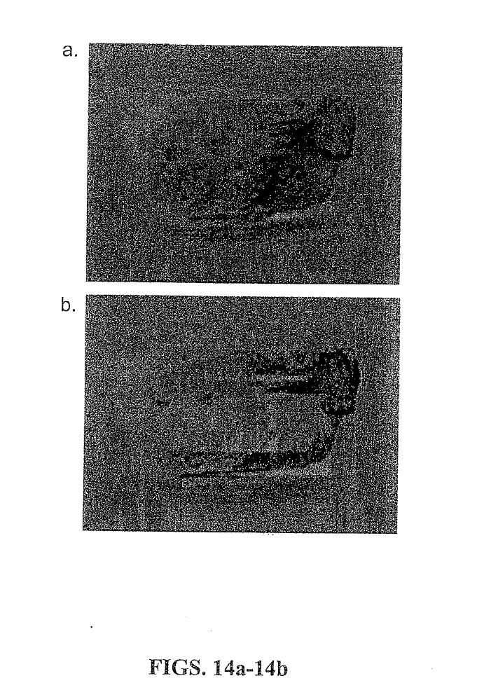

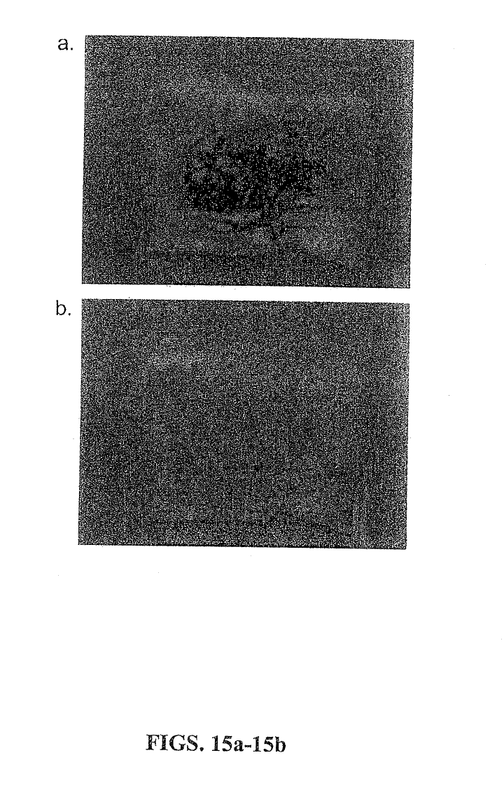

Blood was smeared onto the surface of a tissue sample that has a flat surface, as shown in FIG. 14a, or a curved surface, as shown in FIG. 15a.

The scanning process is started by adjusting the laser focus spot such that the plasma and ablation around the lowest area of the sample can be observed. Each time, after scanning a sample area, the distance between the stage and the lens is increased such that the focus moves up a certain distance and a higher area of the sample is ablated. This process is carried out several times until a layer is ablated from the full sample area. This procedure was carried out on both the flat (relatively) surface sample and the curved surface sample. Results are presented in FIGS. 14 and 15. In some embodiments, to achieve an optimal surface decontamination effect, scanning at different axial positions along the optical axis several times may be necessary.

Application of the USP laser ablated blood from the surface of both the flat and curved tissue samples (see FIGS. 14b and 15b).

### Example 7

### Ablation of Cells Cultured on Slide Surface by USP Laser

USP laser beam was applied to a slide having cells of LNCaP cell line adhered to its surface. LNCaP cells are androgen-sensitive human prostate adenocarcinoma cells. The beam was applied at the parameters shown in Table 11.

TABLE 11
Parameters of USP laser used for ablating cells from a slide surface.

| Pulse Parameter | Setting | |
|---|---|---|
| Duration | 1.1 | ps |
| Energy | 5 | μJ |
| Repetition Rate | 5.05 | kHz |
| Scanning Velocity | 5 | mm/s |
| Wavelength | 1552 | nm |

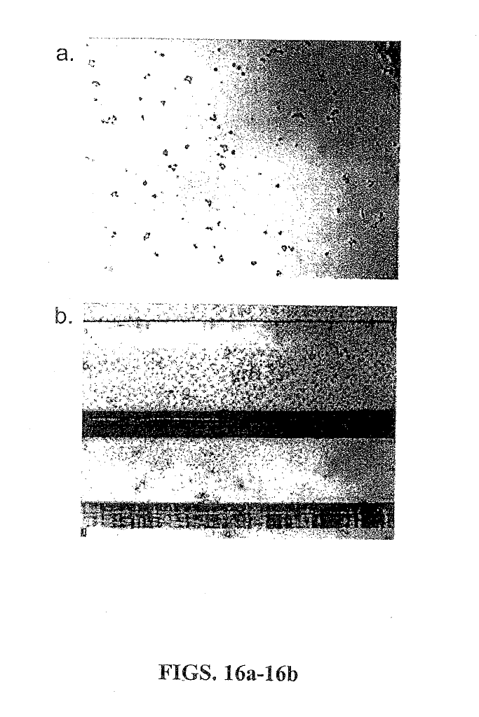

The LNCaP cultured cells were distributed across the surface of the slide as shown in FIG. 16a. Application of the USP laser beam ablated the cells from the surface, as shown in FIG. 16b.

### Example 8

### Ablation of E Coli Cultured on Surface of Agar Plate by USP Laser

USP laser beam was applied to an agar plate cultured with E. coli. The beam was applied at the parameters shown in Table 12.

TABLE 12
Parameters of USP laser used for ablating E. coli
from the surface of an agar plate.

| Pulse Parameter | Setting | |
|---|---|---|
| Duration | 1.1 | ps |
| Energy | 5 | μJ |
| Repetition Rate | 5.05 | kHz |
| Scanning Velocity | 5 | mm/s |
| Wavelength | 1552 | nm |

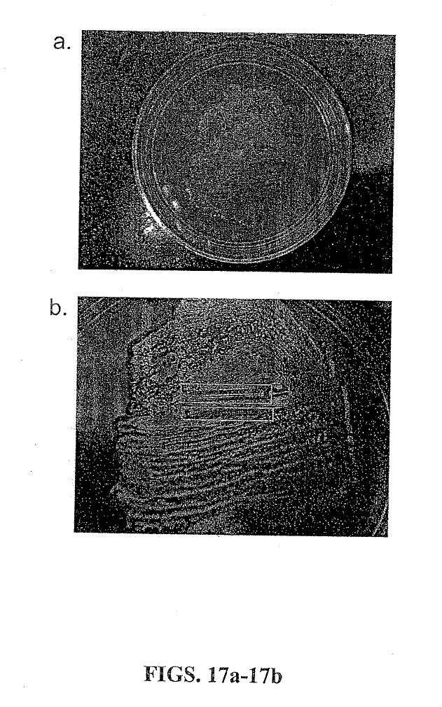

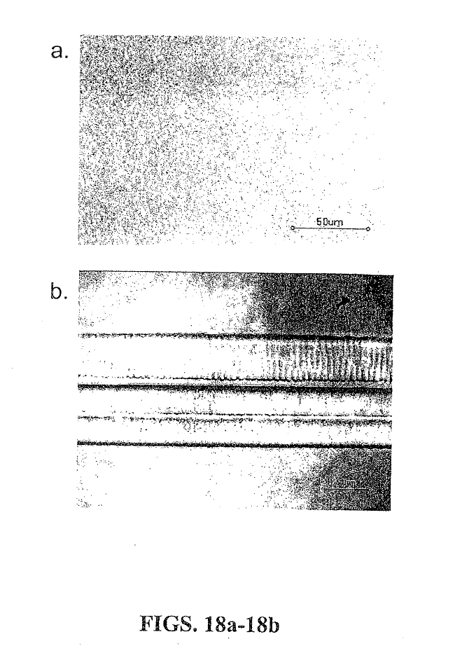

E. coli was cultured on agar plates and were spread by a wire loop throughout the agar plate surface. The agar plates were then incubated for either 12 hours (see FIG. 17a) or 36 hours (see FIG. 18a). Application of the USP laser beam created an ablated area on the surface of agar plates incubated for either duration (see FIGS. 17b and 18b).

### Example 9

### Ablation of an Internal Volume of PDMS by USP Laser

USP laser beam was applied to a sample of PDMS in a direction normal to the PDMS surface to ablate internal material in the sample. The parameters of the USP laser are shown in Table 13.

TABLE 13
Parameters of USP laser used for separating
layers of a sample of PDMS.

| Pulse Parameter | Setting | |
|---|---|---|
| Duration | 1.1 | ps |
| Energy | 1.5 μJ-5 μJ | |
| Repetition Rate | 2 kHz-500 kHz | |
| Scanning Velocity | 20 | mm/s |
| Wavelength | 1552 | nm |

The effects of repetition rate were assessed for two different pulse energies in order to determine the optimal parameters for separating layers of PDMS. The results of the analysis are shown in Table 14.

TABLE 14
Results of the assessment of ablation characteristics
at various repetition rates and pulse energies

| Repetition Rate | 1.5 μJ | 2.0 μJ | 2.5 μJ | 5.0 μJ |
|---|---|---|---|---|
| 2.0 kHz | b, d | b, d | a, d | a, d |
| 5.0 kHz | b, d | a, d | a, d | a, c, e, g |
| 10.0 kHz | b, d | a, c, e, g | a, c, e, g | a, c, f, g |
| 20.0 kHz | a, d | a, c, e, g | a, c, e, g | a, c, f, g |
| 50.0 kHz | a, c, e, g | N/A | N/A | a, c, f, g |
| 100.0 kHz | a, c, e, g | a, c, f, g | a, c, f, g | a, c, f, g |
| 500.0 kHz | N/A | N/A | N/A | a, c, f, h |

(a): ablation generated and can be seen in experiment; obvious ablated shadow after experiments;
(b): no ablation seen or generated;
(c) sample can be separated after experiment;
(d) sample cannot be separated after experiment;
(e): ablated surface does not seem dark;
(f) ablated surface seems dark;
(g) no obvious carbonized particle generated;
(h) obvious carbonized particle generated

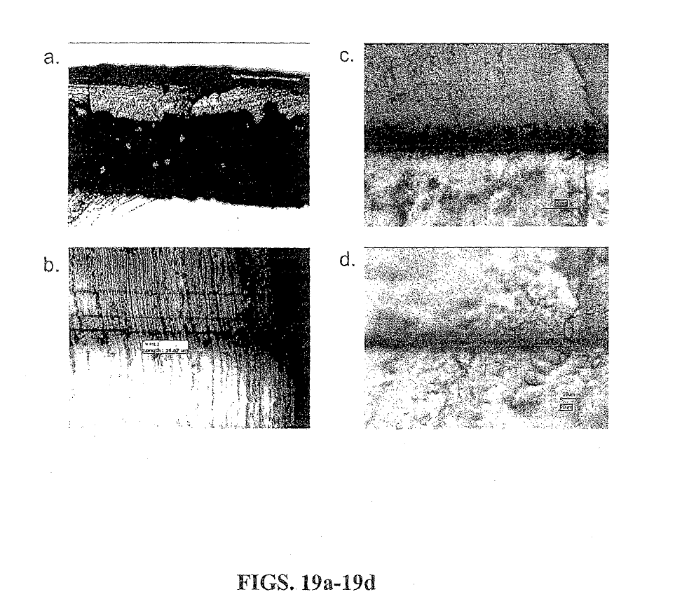

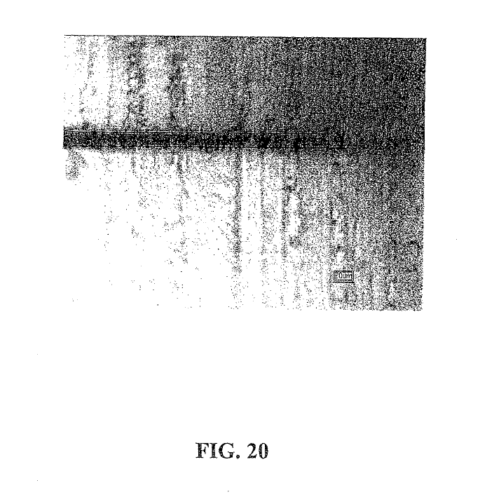

The study revealed that, in general, application of the USP laser at a higher repetition rate and at a greater pulse energy was more effective in ablating material beneath the surface of the sample, and producing a separated layer. In fact, application at a pulse energy of 5.0 μJ was effective in ablating material at all repetition rates, including 500 kHz (see FIG. 19a), 100 kHz (FIG. 19b), 20 kHz (FIGS. 19c), and 5 kHz (FIG. 19d). This is in contrast to, for example, application of USP laser at 2 μJ and 5 kHz, which resulted in no ablation such that the layers could not be separated (see FIG. 20).

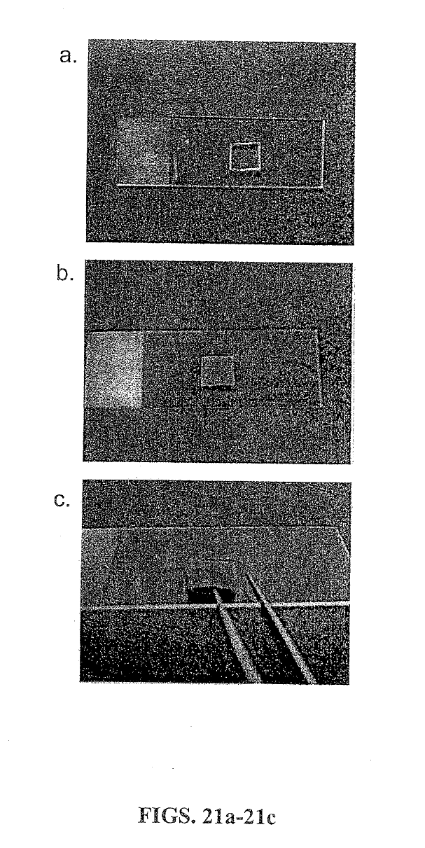

A macroscopic view of the effects of USP laser in separating a layer of PDMS is shown in FIGS. 21a-21c. The dimensions of the PDMS sample were 10 mm×3 mm×4 mm (L×W×D), as shown in FIG. 21a. Application of the USP laser applied at 5 μJ, 100 KHz, and 20 mm/s separated the PDMS sample into layers, as shown in FIGS. 21b and 21c. The separation is more apparent in FIG. 21c, which is a lateral view of the PDMS sample.

USP laser beams can also cut layers of varying thicknesses. In certain embodiments, a USP laser may cut a layer of about 18 μm in thickness. Examples of thicknesses of PDMS layers that can be separated according to the methods of the present invention include thicknesses of about 20 μm, about 25 μm, about 30 μm, about 35 μm, about 40 μm, about 45 μm, about 50 μm, about 60 μm, about 70 μm, and about 80 μm.

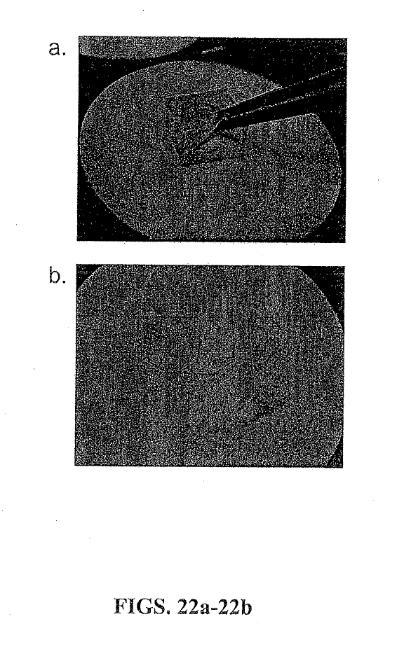

In addition to ablating layers of material, USP laser can also ablate internal volumes in specific shapes and forms. Examples include a V-shaped space inside the PDMS as shown in FIG. 22a, or micro-channels resembling tree branches, as shown in FIG. 22b.

Separation of Skin Tissue into Layers by USP Laser

### Example 10

USP laser beam was applied to an epidermal tissue sample in a direction normal to the tissue surface at the parameters shown in Table 16.

TABLE 16
Parameters of USP laser used for separating
layers of a sample of epidermis.

| Pulse Parameter | Setting | |
|---|---|---|
| Duration | 1.1 | ps |
| Energy | 2 | μJ |
| Repetition Rate | 100 | KHz |
| Scanning Velocity | 20 | mm/s |
| Wavelength | 1552 | nm |

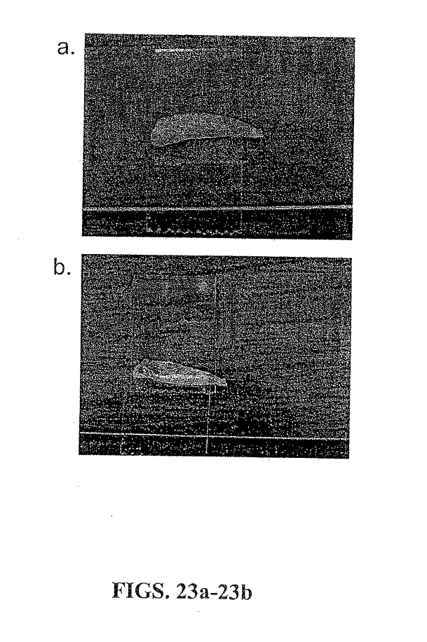

The USP laser partially separated the epidermis sample into layers, as shown in FIGS. 23a-23b. The addition of 70% ethanol to moisten the sample also helped in the application of the laser.

### Example 11

Methods & Materials

Experimental Setup

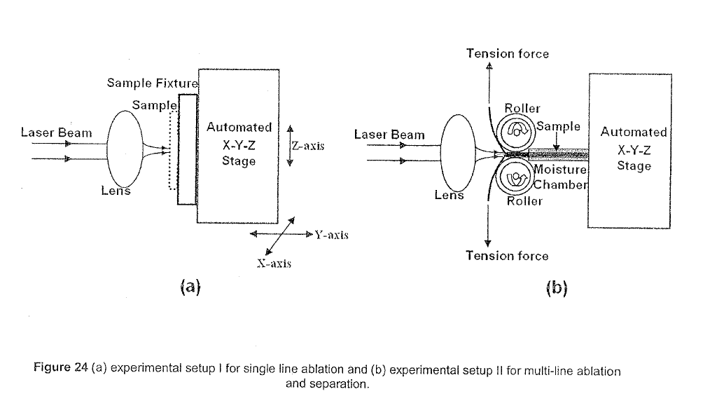

The experimental setup for USP laser tissue ablation in this example is composed of four main parts: a USP laser, a beam delivery system, a work stage and a whole control system. A commercial Erbium doped fiber laser (Raydiance, Inc.) was used in the instrumentation. The laser outputs pulses with repetition rate tunable between 1 Hz and 500 kHz. The output pulse energy is variable from 1 to 5 μJ. The laser central wavelength is 1552 nm and its pulse width is 900 fs. In the beam delivery system, the laser beam was focused to the target through an objective lens (Mitutoyo M Plan Apo NIR 20x, NA=0.40, f_{L}=20 mm) as shown in FIG. 24. The laser beam before the lens is about 10 mm in diameter and the diffraction-limit focal spot diameter (2.44 λf_{L}/D) in free space is estimated as 8 μm. A digital power meter was used to measure the laser power loss in the beam delivery system. It is found that the total loss is about 50%. Such a loss has been accounted for in the irradiation pulse energy values stated hereafter.

The whole control system is a RayOSTM laptop interface which controls the laser output parameters (mainly pulse energy and repetition rate) as well as the motion of the 3-axis precision compact linear stage (VP-25XA, Newport). The work stage for mounting a tissue sample was fixed to the 3-D automated translation stage through which the alignment of optics and laser scanning were realized. There are two designs for the work stage in this example. FIG. 24(a) shows the schematic diagram of experimental setup I with a plate fixture for sample mounting. This setup is simple and was used for characterizing the single line scanning ablation features.

For a wet tissue mounting in this example, in order to avoid deformation of the tissue, a moisture chamber that keeps the tissue wet during the laser processing was utilized as sketched in FIG. 24(b). A tissue feeding and pulling scheme was also designed in experimental setup II as shown in FIG. 24(b) such that the separation interface was always exposed to the laser focal spot through the pulling of two opposite tension forces. Therefore, one is less likely to have to focus the beam into deep tissue, and the strong attenuation of biological tissues against light is less of a consideration. With laser ablation at the exposed interface, the two opposite tension forces pull and split the dermis into two separate layers. An evacuator system (FX225, EDSYN), which is not shown in FIG. 24, was also employed to collect plasma plume residue and debris during the laser processing.

Tissue Samples

In this example, donor dermal tissues were used. The donor skin tissue was processed with a series of soak processing - sodium chloride, triton and finally disinfection soak to get epidermis removed and the processed wet tissue sample was whole dermis. The dermal tissue samples are about 2 mm thick and precut into a dimension about 10 mm long and 5 mm wide, if not otherwise specified in this example.

Like most natural objects the human skins have spectral variability which is in this case mainly due to amount, density, and distribution of melanin. The skin can be described as an optically inhomogeneous material because under the surface there are colorant particles which interact with light, producing scattering and coloration. Light scattering in biological tissues is very strong (see e.g., Troy, T L, Thennadil, S N, J. Biomed. Opt., 6(2):167-176 (2001)). At wavelength 1552 nm, water absorption in wet dermis is also significant; and this may reduce the effect of scattering and improve ablation quality.

Microscopy & Measurements

Immediately following ablation, the micro topography and surface quality of the ablated tissue sample were examined by an upright digital microscope (National Optical DC3-156-S). Then the treated samples were fixed in 2% phosphate buffered glutaraldehyde for 2 hrs, rinsed twice in phosphate buffer and dehydrated in ethanol. After critical point drying and metal coating, the tissue samples were checked by a scanning electron microscopy (SEM) (AMRAY 1830I). For the histological evaluation, the samples were routinely dehydrated in a series of graded ethanol. Then the samples were fixed in paraffin wax and sectioned into 10 μm-thick slices. After that, the slices were stained with Hemaoxylin and Eosin (H&E). Finally the samples were viewed and photographed by a Nikon Eclipse E600 microscope system. The thickness of the separated samples was measured by a vernier caliper.

Results & Discussion

Line Scanning and Ablation Threshold

Among the parameters that affect the ablation are irradiation pulse energy, pulse repetition rate and speed of scanning. The irradiation pulse energy, E that is 50% of the laser output energy, determines whether the incident laser fluence is above the critical value that plasma-mediated ablation occurs. The pulse repetition rate, f, and the moving speed of work stage, s, determine the pulse overlap intensity and can be combined into one parameter—the pulse overlap rate which is equal to f/s.

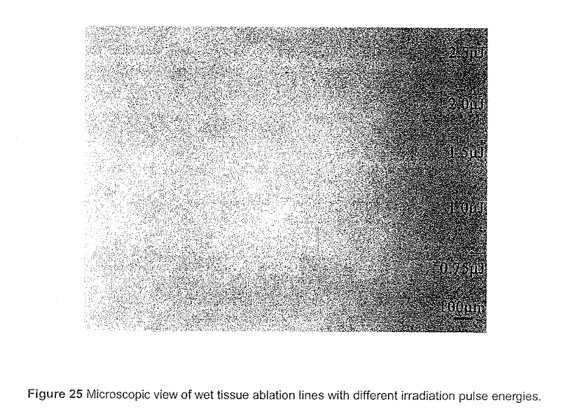

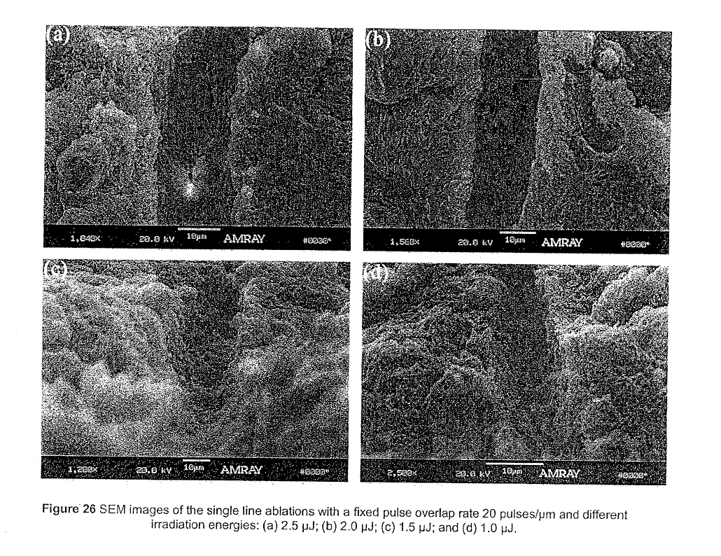

FIG. 25 shows a picture (40× magnification) taken by the digital microscopy for five laser scanned lines on a wet dermis surface with different irradiation pulse energies (0.75 μJ-2.5 μJ). The pulse repetition rate was 500 kHz and the moving speed of the stage was 25 mm/s. Thus, the pulse overlap rate was 20 pulses/μm. The imprints in FIG. 25 reflect the generated ablation lines. The width of the imprints increases as the pulse energy increases and is in the range from 30 to 50 μm.

Fine inspections of the ablation lines are conducted by the SEM measurement and four representative SEM images are shown in FIG. 26 for the four ablation lines generated with irradiation pulse energy 1.0-2.5 μJ, respectively. From the top view images in FIGS. 26(a) and (b), one can measure the average ablation line width as 18.5±1.3 μm and 15.6±0.7 μm for the cases of 2.5 and 2.0 μJ irradiation pulse energies, respectively. While the cut width using mechanical tools such as general surgical blade or scalpel is in the range from 100 μm to 1 mm; thus, the USP laser ablation is more precise and results in less waste. FIGS. 26(c) and (d) are views with a tilt angle for the ablation lines of 1.5 and 1.0 μJ irradiation pulse energies, respectively. It is seen that the dermis surface is not very flat and has a roughness of about 5 μm. It is thought that this roughness will enhance light scattering on the surface and affect the effective size of the beam focal spot at dermis surface.

In laser ablation, the effective radius, r_{eff}, of the focal spot can be found by the slope of the following formula (see Baudach, S et al. Appl. Phys. A 1999, 69:S395-8):

$${D}^{2} = 2 {r}_{eff}^{2} ln ( \frac{{F}_{0}}{{F}_{th}} ) , \qquad ( 1 )$$

where D is the diameter of the ablation crater and Fₜₕ is the ablation threshold fluence.

For laser pulses with a Gaussian spatial beam profile, the maximum irradiation fluence F₀ can be calculated from the irradiation pulse energy E as

$${F}_{0} = \frac{2 E}{π {r}_{eff}^{2}} . \qquad ( 2 )$$

An ablation line comprises continuously ablated craters along the laser scanning direction. When the pulse overlap rate is so intense that no individual crater can be distinguished (such as displayed in FIG. 26), the ablation line width is then equivalent to the diameter of the ablated crater generated by N repeated pulses. The equivalent pulse number can be approximated by

N=2r_{eff}f/s (3)

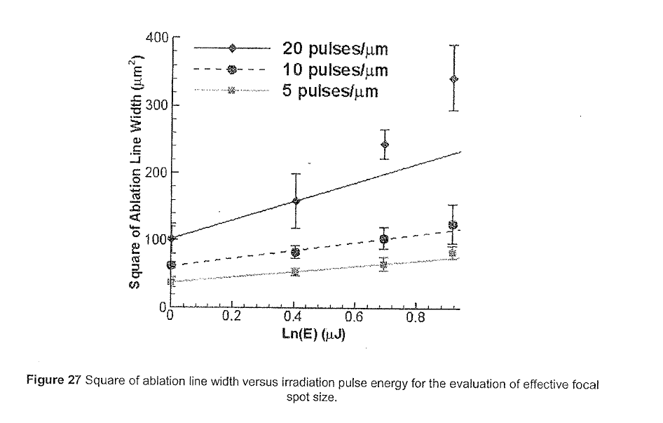

FIG. 27 plots the relationship—the square of the ablation line width versus the logarithm of irradiation pulse energy for three different pulse overlap rates. In Bonse et al. Appl. Phys. A 2001, 72:89-94, it is pointed out that the data at high fluence points should be excluded from linear fitting because the deviation of the intensity from the Gaussian distribution at the “edge” of high fluence beam will lead to nonlinearity. It is thought that the accumulated fluence for the ablation lines of this example is very high because the equivalent pulse number N is very large as calculated in Table 1. Thus, only low fluence points are adopted for the linear fitting to obtain the slopes of the three curves in FIG. 27, in particular for the curve with 20 pulses/μm pulse overlap rate. The calculated effective radii for the focal spots with different pulse overlap rates and the corresponding equivalent pulse number are listed in Table 1.

TABLE 1
Effective focal spot radii and ablation thresholds
for different pulse overlap rates.

| | Pulse overlap rate (pulses/μm) | | |
|---|---|---|---|
| | 5 | 10 | 20 |
| Equivalent pulse number | 45 | 110 | 336 |
| Effective focal spot radius (μm) | 4.5 | 5.5 | 8.4 |
| Ablation threshold Fₜₕ (J/cm²) | 1.27 | 0.75 | 0.43 |

It is seen that the effective spot size (9-17 μm) is bigger than the diffraction-limit spot size (8 μm) in free space. This may be attributed to the strong scattering of light on the rough dermis surface. When the pulse overlap rate is just 5 pulses/μm, it is seen that the calculated effective radius is close to the diffraction-limit prediction. With increasing pulse overlap rate, the accumulated fluence increases and the deviation between the calculated effective radius and the diffraction-limit prediction widens.

After obtaining the effective focal radius, the fluence can be calculated by equation (2) and the thresholds for different pulse overlap rates can be acquired by extending the fitted lines in FIG. 27 to intersect with the abscissa. Table 1 also lists the ablation thresholds for the three different pulse overlap rates. An accumulation model is given as

Fₜₕ(N)=Fₜₕ(1)N^{ξ-1}, (4)

where Fₜₕ(1) and Fₜₕ (N) refer to the ablation threshold due to a single pulse and N pulses, respectively. The exponent ξ is the so-called incubation factor. Using the data in Table 1, a least-squares fitting line of ln(NFₜₕ(N)) versus ln(N) can be drawn and the slope yields an incubation factor ξ=0.46±0.03. Therefore, the ablation threshold for the wet human dermis in this example is determined as Fₜₕ(1)=9.65±1.21 J/cm2. The uncertainties are obtained using the methods described in Higbie, J, Am. J. Phys. 1991, 59(2):184-5 and Holman, J P, Experimental methods for engineers, 7^{th} ed. Boston: McGraw Hill (2001).

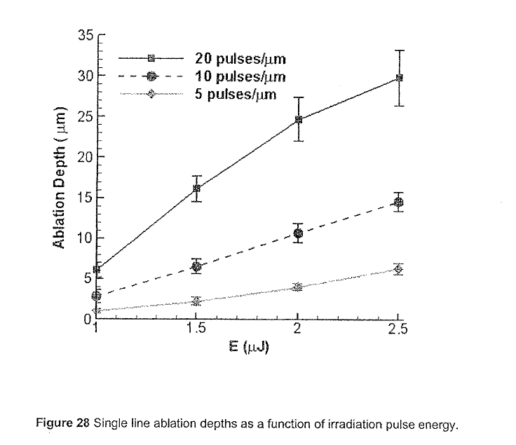

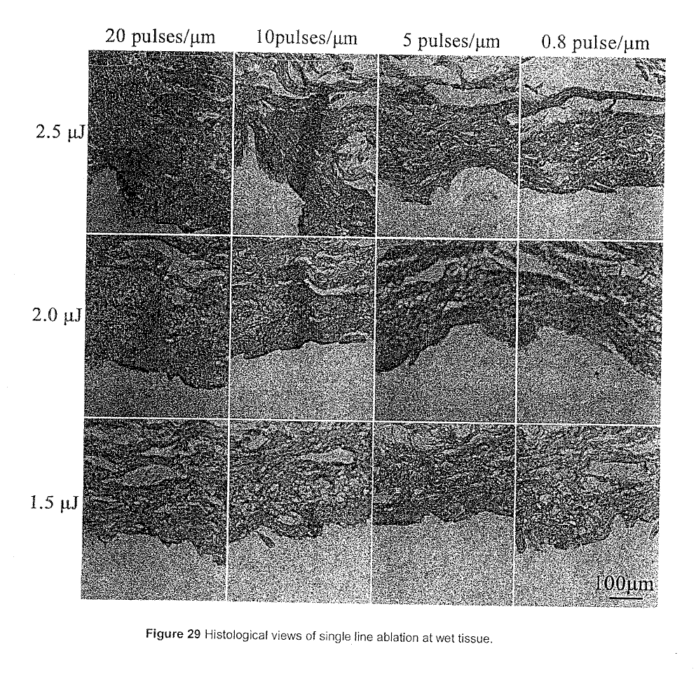

FIG. 28 shows the ablation depth change with the irradiation pulse energy for different pulse overlap rates in the situation of single line scanning ablation. For each ablation depth datum, three samples were measured to obtain the average value and the uncertainty. Care should be taken in the preparation of fixing and drying the tissue samples for SEM examination, as the samples may become somewhat distorted, and the distortion may affect the measurement accuracy as well. From FIG. 28 it is seen that the ablation depth increases with both the irradiation pulse energy and overlap rate. Pulse overlap rate increases with the pulse repetition rate but decreases with the scanning speed, and the ablation progress is linearly proportional to the scanning speed. For a fixed scanning speed, the ablation production efficiency increases with increasing pulse energy and repetition rate.

Histological Evaluations

In order to examine the degree of thermal damage, the histology of some line scanning ablated samples was analyzed. FIG. 29 shows the sectional view (200× magnification) of 12 H&E stained wet dermis samples ablated with single line surface scanning with different laser parameters. The selected pulse energies are 1.5, 2.0 and 2.5 μJ, respectively. The pulse overlap rates are 0.8, 5, 10 and 20 pulses/μm, respectively. The irradiation surface in the pictures faces down and the beam spot is around the middle in each picture. Thermal damaged zone is visualized by the shadow area, because the elastic fibers in the damaged zone are no longer apparent, having been converted into an amorphous, coagulated mass. As observed in FIG. 29, no thermal damage or structure change occurs in the &inns when the pulse overlap rate is 5 pulses/μm and below, even in the case of high irradiation pulse energy (2.5 μJ). When the pulse overlap rate is 10 pulses/μm and above, however, a clear thermal damage zone is observed, in particular when the pulse energy is 2.0 μJ and above. The higher the pulse overlap rate or the higher the pulse energy, the larger and the severer (darker) is the thermal damaged zone. In certain embodiments, in order to minimize or eliminate thermal damage, operation with a lower pulse overlap rate is preferred. In this example, a pulse overlap rate of up to 5 pulses/μm is preferred for single line ablation.

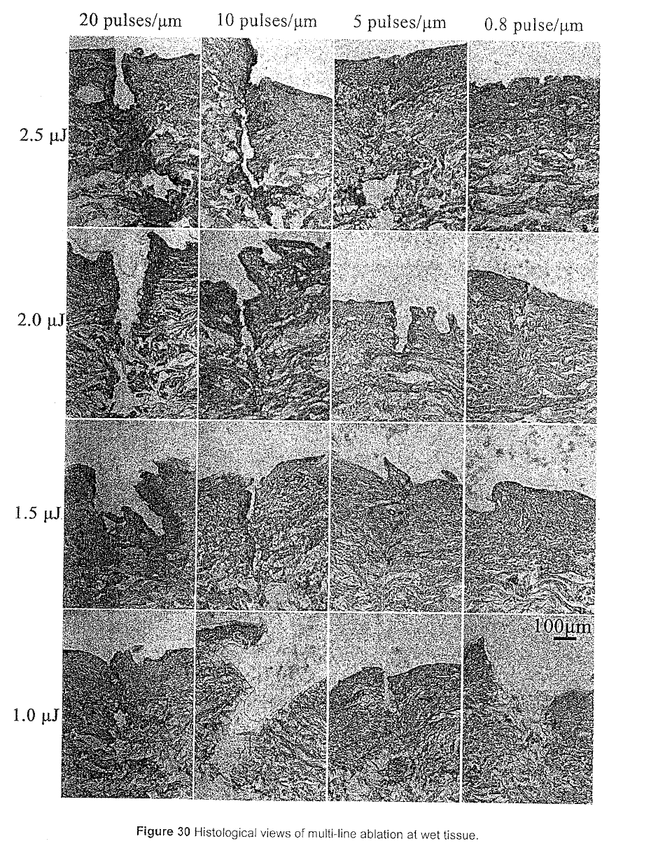

In FIG. 28, ablation scanning of multiple lines is conducted to achieve tissue separation or cutting. Some representative histological results of multi-line ablation are presented in FIG. 30 for evaluation and comparison, where the sectional views (200× magnification) of 16 ablation processed tissue samples with different laser parameters are illuminated. Each tissue sample was repeatedly line scanned for 100 times using experimental setup II. During the processing, the ablation interface was always renewed via the tension through the two opposite tension forces. The selected pulse energies are 1.0, 1.5, 2.0 and 2.5 μJ, respectively. The pulse overlap rates are 0.8, 5, 10 and 20 pulses/μm, respectively. The irradiation surface in each picture faces up and the beam focal spot is around the middle. Clear cuts to a certain depth in all the samples are observed.

Table 2 summarizes the sizes of the lateral thermal damage zone around the cut edge for the laser parameter sets considered in FIG. 30. The thermal damage behavior for the multi-line scanning cases is very similar to that observed in the single line scanning results, even though the accumulated fluence in multi-line scanning is 100 times stronger than the single line scanning. It is thought that the reason for this is that between two successive scans, the lateral accumulation of thermal energy is trivial because the energy has been dissipated into the surroundings. In the cases of 100-line ablation with pulse overlap rate 5 pulses/μm, lateral thermal damage is observable within a 10 μm zone when the irradiation pulse energy is 2.0 μJ or above, and the damage is reduced to 2-3 μm when the irradiation pulse energy is below 2.0 μJ. It is believed that this is because the accumulated energy between two successive scans has not been fully dissipated yet. This may be addressed by delaying the repeated scanning time.

TABLE 2
Lateral thermal damage zones resulted from 100-line ablation.

| | f/s | | | |
|---|---|---|---|---|
| E | 20 pulses/μm | 10 pulses/μm | 5 pulses/μm | 0.8 pulse/μm |
| 2.5 μJ | 67 ± 10 μm | 40 ± 8 μm | 8 ± 3 μm | 6 ± 4 μm |
| 2.0 μJ | 54 ± 10 μm | 38 ± 8 μm | 6 ± 4 μm | 4 ± 4 μm |
| 1.5 μJ | 52 ± 8 μm | 18 ± 5 μm | 3 ± 2 μm | 3 ± 3 μm |
| 1.0 μJ | 26 ± 6 μm | 3 ± 2 μm | 2 ± 2 μm | 2 ± 2 μm |

Apart from the qualitative examination, FIG. 30 also shows the cut (ablation) depths for different pulse overlap rates and pulse energies. It is seen that the ablation depth generally increases as the pulse energy and/or overlap rate increase. In this example, two spring steel clips (SBC-78210) were used as the tension forces and the forces were not optimized in line with the single line ablation depth. Thus, the cutting depth due to multi-line ablation is not a simple multiplication of corresponding single line ablation depth. For example, the 100-line ablation depth for the picture in FIG. 30 with pulse overlap rate 5 pulses/μm and irradiation pulse energy 2.0 μJ is only 210 μm although its single line ablation depth from FIG. 28 reaches to 4.0 μm. Without being bound to theory, reasons that may contribute to degrade the multi-line ablation depth include beam block by the edges of the prior ablation grooves or by the generated residues and debris and beam alignment. In addition, as noted earlier, during preparation of samples for histological view, the samples could become somewhat distorted, and this may affect measurement accuracy as well.

Since the cutting efficiency is directly proportional to the ablation depth and scanning speed, it is desirable to operate the laser tissue processing system at high irradiation pulse energy, high pulse repetition rate and high speed of scanning. At the same time, it is desirable that the pulse overlap rate is controlled to avoid thermal damage.

In certain embodiments, for wet dermis cutting and separation, USP operation parameters are as follows: irradiation pulse energy=1.5 μJ, stage moving speed=25 mm/s (the maximum of the current instrument), pulse repetition rate=125 kHz, and pulse overlap rate=5 pulses/μm.

Tissue Separation

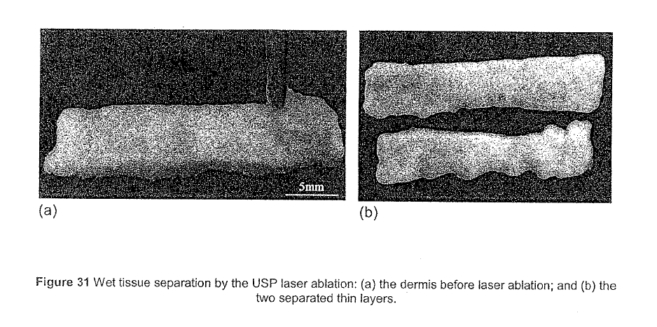

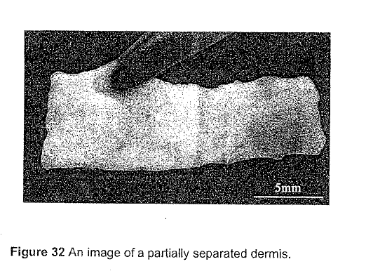

The USP laser thin layer separation of wet dermis in this example is demonstrated in FIGS. 31 and 32. FIG. 31(a) shows one original wet dermis sample before laser ablation. The sample was 30 mm long, 8 mm wide and 1.4 mm thick. Then the tissue was processed with experimental setup II with pulse overlap rate 5 pulses/μm and pulse energy 1.5 μJ. FIG. 31(b) shows the two separated layers that are about 500 μm and 800 μm thick with about 10% unevenness, respectively for the upper and lower pieces.

The separated dermis layers can be further split. FIG. 32 shows the partially separated result of another dermis layer of 20 mm long, 6 mm wide and 560 μm thick with the same laser parameters. The thickness of the further separated dermis thin layer is about 220 μm with about 20 μm unevenness. Upon inspection of the separated layers in FIGS. 31 and 32, no severe thermal damage like charring or melting was found. In certain embodiments, adjustments to keep the tissue moving and addition of water to the moisture chamber may be made during the processing.

Table 3 lists several dermis tissue separation results. The separated layers have a uniform thickness with less than 10% uncertainty.

TABLE 3
Results from several dermis separation tests.

| Sample | Thickness of the original dermis | Thickness of the separated |
|---|---|---|
| No. | (mm) | thinner layer (mm) |
| 1 | 0.56 | 0.22 ± 0.02 |
| 2 | 0.60 | 0.23 ± 0.02 |
| 3 | 0.80 | 0.32 ± 0.02 |
| 4 | 0.80 | 0.33 ± 0.02 |
| 5 | 1.40 | 0.50 ± 0.03 |
| 6 | 2.00 | 0.56 ± 0.03 |

While specific embodiments of the subject invention have been discussed, the above specification is illustrative and not restrictive. One skilled in the art will appreciate that numerous changes and modifications can be made to the invention, and that such changes and modifications can be made without departing from the spirit and scope of the invention. The full scope of the invention should be determined by reference to the claims, along with their full scope of equivalents, and the specification, along with such variations.

Each patent, patent application, and publication cited or described in the present application is hereby incorporated by reference in its entirety as if each individual patent, patent application, or publication was specifically and individually indicated to be incorporated by reference.

1.-25. (canceled)

26. A method of cutting a biological tissue, comprising applying an ultrashort pulse (USP) laser to the tissue.

27. The method of claim 26, wherein the pulses have a duration of about 100 fs to about 50 ps, a repetition rate of about 1 Hz to about 500 kHz, a pulse energy of about 1 to about 100 μJ, and a wavelength of between about 776 nm and 1552 nm.

28. The method of claim 26, further comprising applying the USP laser to the tissue until the tissue is separated in two or more portions.

29. The method of claim 26, further comprising (i) focusing the beam to the biological tissue at different depths, wherein the focused beam induces optical breakdown and ablates the biological tissue at the focused site; (ii) repeating the application of the focused beam to the biological tissue in a plurality of sites through the depth of the biological tissue, wherein the focused beam ablates the biological tissue at the plurality of sites.

30. The method of claim 26, wherein the biological tissue is selected from the group consisting of allograft, xenograft, autograft, and biologic matrix.

31. The method of claim 30, wherein the allograft, xenograft, or autograft is selected from the group consisting of dermal tissue, musculoskeletal tissue, cardiovascular tissue, connective tissue, and neural tissue.

32. The method of claim 31, wherein the allograft, xenograft, or autograft is dermal tissue.

33. The method of claim 30, wherein the biologic matrix is an acellular dermal matrix.

34. The method of claim 26, wherein the biological tissue is selected from the group consisting of bone, muscle, fascia, bladder, stomach, heart, small intestine, large intestine, and parenchymal organs.

35. A method of cutting a biological tissue, comprising:
(i) providing a biological tissue;
(ii) generating a laser beam of ultrashort pulses, wherein the pulses have a duration of about 100 fs to about 50 ps, a repetition rate of about 1 Hz to about 500 kHz, a pulse energy of about 1 to about 100 μJ, and a wavelength of between about 776 nm and 1552 nm;
(iii) applying and focusing the beam to the biological tissue at different depths, wherein the beam is in a direction normal to the surface, and wherein the focused beam induces optical breakdown and ablates the biological tissue at the focused site;
(iv) repeating the application of the focused beam to the biological tissue in a plurality of sites through the depth of the biological tissue, wherein the focused beam ablates the biological tissue at the plurality of sites, thereby cutting the tissue.

36.-63. (canceled)

64. A method of precision ablating unwanted material from an area on a surface of a biological tissue, comprising applying an ultrashort pulse (USP) laser to the surface of the tissue, wherein the laser does not induce damage to the tissue below the unwanted material.

65. The method of claim 64, further comprising:
(i) generating a laser beam of ultrashort pulses, wherein the pulses have a duration of about 100 fs to about 50 ps, a repetition rate of about 1 Hz to about 500 kHz, a pulse energy of about 1 to about 100 μJ, and a wavelength of between about 776 nm and 1552 nm;
(ii) focusing the beam to the surface of the biological tissue at a first site with a focus spot size in the range of 2-10 μm, wherein the beam is in a direction normal to the surface of the tissue and to a depth of the unwanted material, and wherein the focused beam induces optical breakdown and removes the unwanted material at the first site via laser-induced plasma ablation; and
(iii) repeating the application of the focused beam to the surface of the biological tissue at a plurality of sites across the surface of the biological tissue, wherein: (a) the focused beam ablate the unwanted material at the plurality of sites, (b) the plurality of sites are adjacent to each other, and (c) the plurality of sites form an area.

66. The method of claim 64, wherein the USP laser is applied in a direction normal to the surface of the transverse laser.

67. The method of claim 64, wherein the USP laser is applied in a direction parallel to the surface of the transverse layer.

68. The method of claim 64, further comprising applying a diagnostic laser beam to the surface of the biological tissue to determine the depth of the unwanted material.

69. (canceled)

70. The method of claim 64, wherein the unwanted material is selected from the group consisting of gram positive bacteria, gram negative bacteria, spore-forming bacteria, yeasts, and fungi.

71. The method of claim 64, wherein the unwanted material comprises a layer of cells.

72. The method of claim 64, wherein the unwanted material comprises residual skin hairs.

73. The method of claim 64, wherein the biological tissue is selected from the group consisting of allograft, xenograft, autograft, and biologic matrix.

74. The method of claim 73, wherein the allograft, xenograft, or autograft is selected from the group consisting of dermal tissue, musculoskeletal tissue, cardiovascular tissue, connective tissue, and neural tissue.

75. The method of claim 73, wherein the biologic matrix is an acellular dermal matrix.

76. The method of claim 64, wherein the biological tissue is selected from the group consisting of bone, muscle, fascia, bladder, stomach, heart, small intestine, large intestine, and parenchymal organs.

77.-99. (canceled)
